(19) United States
(12) Patent Application Publication
(10) Pub. No.: US 2018/0338955 A1
(43) Pub. Date: Nov. 29, 2018

(54) METHODS FOR REGULATION AND TREATMENT OF GLIOMA CELL MIGRATION AND GLIOBLASTOMA

(71) Applicants: Nikolaos Tapinos, Barrington, RI (US); Atom Sarkar, Danville, PA (US); Jennifer Ness-Myers, Selinsgrove, PA (US)

(72) Inventors: Nikolaos Tapinos, Barrington, RI (US); Atom Sarkar, Danville, PA (US); Jennifer Ness-Myers, Selinsgrove, PA (US)

(21) Appl. No.: 15/772,290

(22) PCT Filed: Oct. 28, 2016
(86) PCT No.: PCT/US2016/059423

§ 371 Date: Apr. 30, 2018

Related U.S. Application Data
(60) Provisional application No. 62/248,108, filed on Oct. 29, 2015.

(51) Int. Cl. A61K 31/4196; C12Q 1/68; G01N 33/53

(52) U.S. Cl. CPC A61K 31/4196; G01N 33/53; C12Q 1/68

(57) ABSTRACT

Methods for treating glioblastoma in a subject comprising administering to the subject an inhibitor of a Src family kinase. Methods for inhibiting the migration of a glial cell comprising contacting the glial cell with an inhibitor of a Src family kinase or reducing the expression of a Src family kinase in the glial cell. Methods for reducing or inhibiting tumor formation in a subject comprising administering to the subject an inhibitor of a Src family kinase or administering an oligonucleotide or a gene editing system that reduces the expression of a Src family kinase within a cell. Ex vivo co-culture systems comprising a Dorsal Root Ganglion (DRG) axon-oligodendrocyte and a gliomal cell. Methods for identifying an inhibitor of glial cell migration or movement. Methods for treating glioblastoma and for isolating pseudopodia of a gliomal cell and for identifying RNA present therein.

### BACKGROUND

Recently, the Cancer Genome Atlas (TCGA) consortium published extensive genetic analysis of the DNA mutation landscape of human glioblastoma (Brennan et al., 2013; Cancer Genome Atlas Research, 2008, which is incorporated by reference herein in its entirety). In addition, it is widely appreciated that human glioblastoma is one of the most aggressive and lethal human tumors due to the presence of tumor-propagating glioma stem cells (GSCs) and the highly migratory nature of these cells (Bao et al., 2006; Beier et al., 2007; Lee et al., 2006; Singh et al., 2003; Vescovi et al., 2006, each of which is incorporated by reference herein in its entirety). Recent studies have revealed extensive intratumoral heterogeneity of human glioblastomas an attribute that contributes to treatment failure and tumor recurrence (Bedard et al., 2013; Patel et al., 2014, each of which is incorporated by reference herein in its entirety).

However, despite the wealth of knowledge regarding the genetic background of this disease, studies on glioma cell migration are hindered by the lack of efficient in vitro or in vivo migration models. Until now, studies on glioma cell migration were performed on Laminin coated surfaces (Joy et al., 2003; Kislin et al., 2009, each of which is incorporated by reference herein in its entirety), collagen and astrocyte layers (Aubert et al., 2008, which is incorporated by reference herein in its entirety), extracellular matrix matrigel layers (Kaur et al., 2012, which is incorporated by reference herein in its entirety), electrospan nanofibers (Rao et al., 2013, which is incorporated by reference herein in its entirety), or postmortem in mouse xenograft models (Kaur et al., 2012, which is incorporated by reference herein in its entirety). Even though all these studies have provided information on the migratory properties and mechanisms of human glioma cells, the real-time interaction of human GSCs with myelinated and non-myelinated axons in the brain has not been studied thus far. In addition, migration of human GSCs occurs in the brain parenchyma through continuous interaction with axon fibers, glial cells, microglia and endothelial cells, which affect the migratory efficiency of GSCs and cannot be accounted for with existing monolayer migration models.

### SUMMARY

Described herein is a Dorsal Root Ganglion (DRG) axon-oligodendrocyte co-culture method to study in real time the migration and interaction of GSCs with axons, which occurs through the extensive formation of pseudopodia. Isolation of pseudopodia-localized RNA followed by eIF4 RNA-IP reveals local transcripts of Lck, Paxillin, Crk-II, and Rac1 that undergo eIF4-dependent translation. Inhibition of Lck blocks the activation of this pathway, the formation of pseudopodia and the migration of GSCs. In vivo administration of a highly-specific Lck inhibitor using an orthotopic xenograft mouse model results in significant inhibition of tumor formation and GSC migration. Targeting this locally-translated Lck-dependent pathway constitutes a novel treatment paradigm for glioblastomas, including treatment in humans.

Also described herein is a method for treating glioblastoma in a subject comprising administering to the subject an inhibitor of a Src family kinase, wherein the inhibitor is not Dasatinib. In some embodiments, the Src family kinase is lymphocyte-specific protein tyrosine kinase (Lck). In certain embodiments, the inhibitor is a small molecule. In some embodiments, the inhibitor reduces the level of phosphorylation of the Src family kinase. In certain embodiments, the inhibitor reduces the level of phosphorylation of paxillin. In some embodiments, the inhibitor is A770041. A “subject” may be a human, mouse, rat, or dog.

Also described herein is a method for inhibiting the migration of a glial cell comprising contacting the glial cell with an inhibitor of a Src family kinase, wherein the inhibitor is not Dasatinib. In some embodiments, the glial cell is a glioma stem cell (GSC). In some embodiments, the number of pseudopodia formed in the glial cell contacted with the inhibitor is reduced compared to the number of pseudopodia formed in a migrating glial cell not contacted with the inhibitor. In certain embodiments, the Src family kinase is lymphocyte-specific protein tyrosine kinase (Lck). In some embodiments, the inhibitor is a small molecule. In certain embodiments, the inhibitor reduces the level of phosphorylation of the Src family kinase. In some embodiments, the inhibitor reduces the level of phosphorylation of paxillin. In certain embodiments, the inhibitor is A770041.

Also described herein is a method for reducing or inhibiting tumor formation in a subject comprising administering to the subject an inhibitor of a Src family kinase, wherein the inhibitor is not Dasatinib. In some embodiments, the tumor is a glioblastoma. In certain embodiments, the Src family kinase is lymphocyte-specific protein tyrosine kinase (Lck). In some embodiments, the inhibitor is a small molecule. In certain embodiments, the inhibitor reduces the level of phosphorylation of the Src family kinase. In certain embodiments, inhibitor reduces the level of phosphorylation of paxillin. In some embodiments, the inhibitor is A770041. In certain embodiments, the subject is a mammal. In some embodiments, the mammal is a dog, mouse, rat, or human.

Also described herein is an ex vivo co-culture system comprising a Dorsal Root Ganglion (DRG) axon-oligodendrocyte and a gliomal cell. In some embodiments, the gliomal cell is a glioma stem cell (GSC). In certain embodiments, the DRG axon-oligodendrocyte is myelinated. In some embodiments, the gliomal cell expresses green fluorescent protein (GFP). In certain embodiments, the GFP is introduced into the gliomal cell via viral infection. In some embodiments, the viral infection comprises a Lentivirus.

Also described herein is a method for identifying an inhibitor of glial cell migration or movement comprising contacting a glial cell in an ex vivo co-culture system with a putative glial cell migration or movement inhibitor; imaging the migration or movement of the glial cell; and comparing the migration or movement of the glial cell contacted with the putative inhibitor to the migration or movement of a glial cell in the co-culture system that has not been contacted with the putative inhibitor, wherein a putative inhibitor that reduces or prevents migration or movement of the glial cell is characterized as an inhibitor of glial cell migration or movement. In some embodiments, the co-culture system comprises a Dorsal Root Ganglion (DRG) axon-oligodendrocyte. In certain embodiments, the cell migration or movement of the glial cell is measured by quantifying the surface area covered by the glial cell. In some embodiments, the quantification of the surface area comprises detection of electrical impedance. In certain embodiments, the glial cell is a gliomal cell. In some embodiments, the gliomal cell is a glioma stem cell (GSC). Also described herein is a method for treating glioblastoma in a subject comprising administering to the subject an inhibitor of glial cell migration or movement identified by the method described above.

Also described herein is a method for imaging the migration or movement of a glial cell comprising live imaging a glial cell present in an ex vivo co-culture system. In some embodiments, the co-culture system comprises a Dorsal Root Ganglion (DRG) axon-oligodendrocyte. In certain embodiments, the live imaging comprises time-lapse photography. In some embodiments, the glial cell is a gliomal cell. In certain embodiments, the gliomal cell is a glioma stem cell (GSC).

Also described herein is a method for isolating pseudopodia of a gliomal cell, the method comprising seeding the gliomal cell on a well comprising pores and fibronectin; culturing the gliomal cell in the well; and isolating pseudopodia of the gliomal cell from the sides of the well. In some embodiments, the pores are at least about 1 μm. In certain embodiments, the pores are about 1 μm. In some embodiments, the fibronectin is human fibronectin. In certain embodiments, the pseudopodia are scraped from the sides of the well. In some embodiments, the well comprises a Boyden chamber. In certain embodiments, the gliomal cell is a glioma stem cell (GSC).

Also described herein is one or more isolated pseudopodia derived from a gliomal cell. In some embodiments, the gliomal cell is a glioma stem cell (GSC). In certain embodiments, the pseudopodia do not express cytoplasmic marker Grp75. In some embodiments, the pseudopodia are isolated by a method comprising seeding the gliomal cell on a well comprising pores and fibronectin; culturing the gliomal cell in the well; and isolating pseudopodia derived from the gliomal cell from the sides of the well.

Also described herein is a method of identifying RNA present in the pseudopodia of a gliomal cell, the method comprising isolating the pseudopodia of the gliomal cell according to the method of any one of claims 43-49 and isolating RNA from the isolated pseudopodia. In some embodiments, at least a portion of the isolated RNA is characterized by a process comprising real-time polymerase chain reaction (PCR). In certain embodiments, at least a portion of the RNA is characterized by a process comprising RNA immunoprecipitation. In some embodiments, the RNA immunoprecipitation is performed on a eukaryotic initiation factor 4 (eIF4) protein.

Also described herein is a method for treating glioblastoma in a subject comprising reducing the expression of a Src family kinase protein in a gliomal cell of the subject. Also described herein is a method for inhibiting the migration of a glial cell comprising reducing the expression of a Src family kinase protein in the glial cell. Also described herein is a method for reducing or inhibiting tumor formation in a subject comprising reducing the expression of a Src family kinase protein in a gliomal cell of the subject. In some embodiments, the Src family kinase protein is lymphocyte-specific protein tyrosine kinase (Lck). In certain embodiments, the expression of the Src family kinase protein is reduced by a method comprising administering an antisense oligonucleotide to the gliomal cell of the subject. In some embodiments, the antisense oligonucleotide targets a transcript encoding the Src family kinase protein. In certain embodiments, the expression of the Src family kinase protein is reduced by a method comprising RNA interference in the gliomal cell of the subject or the glial cell. In some embodiments, the RNA interference method comprises administering a small interfering RNA (siRNA) to the gliomal cell of the subject or the glial cell. In certain embodiments, the siRNA targets a transcript encoding the Src family kinase protein. In some embodiments, the RNA interference method comprises administering a small hairpin RNA (shRNA) to the gliomal cell of the subject or the glial cell. In certain embodiments, the shRNA targets a transcript encoding the Src family kinase protein. In some embodiments, the expression of the Src family kinase protein is reduced by a method comprising gene editing. In certain embodiments, the gene editing method comprises altering the expression of a gene that encodes the Src family kinase protein. In some embodiments, the gene editing method comprises administering a clustered regularly interspaced short palindromic repeats (CRISPR)/CRISPR-associated (Cas) (CRISPR/Cas) system to the gliomal cell of the subject or the glial cell.

### BRIEF DESCRIPTION OF THE DRAWINGS

FIG. 1 shows that human GSCs form tumor-like structures and migrate along myelinated axonal tracks on an ex vivo co-culture system as described herein. FIG. 1A is a representative picture of human GSCs forming GFAP^{+(red)}/Ki67^{+(green)} tumor-like structures on DRG axons. FIG. 1B shows the establishment of myelinated MBP^{+(green)} axonal tracks after addition of oligodendrocyte progenitor cells on purified DRG axons (stained red with Neurofilament antibody). FIG. 1C is a representative picture of human GSCs stained with human Mitochondrial marker (Mito^{+(green)}) preferentially migrating along myelinated axonal tracks (MBP^{+(red)}). Scale bars: 200 μm.

FIG. 2 shows that human glioma cells interact with axons through the formation of pseudopodia. FIG. 2A shows that human GSCs were seeded on purified DRG axons and live cell imaging was performed with images acquired every 10 minutes using a Zeiss Axiovert microscope equipped with the AxioVision Software. Dynamic extension and retraction of pseudopodia were seen (arrowheads) as the GSCs migrated along the axons. FIG. 2B shows that human GSCs were seeded in Boyden chambers separated by a membrane with 1μ pore openings and allowed to migrate towards a 10% serum gradient at the lower chamber. Confocal z-stack images of the chambers show that only the phalloidin^{+(red)} GSC pseudopodia cross the 1μ pore while the DAPI nuclei and the rest of the cell body remain on the upper chamber (Left panel). Protein isolation from the upper and lower chambers shows that the pseudopodia fraction is pure and does not express the cytoplasmic marker Grp75 (Right panel).

FIG. shows localized expression and translation of migration specific transcripts in hGSC pseudopodia. FIG. 3A depicts RNA from purified pseudopodia of hGSCs from two patients representing mesenchymal (GSC1) and classical (GSC2) subtypes of glioblastomas used to perform a real-time PCR array for migration specific genes (SA Biosciences). Heat map expression analysis shows that RNA isolated from pseudopodia of human GSCs contains various migration specific transcripts including Lck, Paxillin, CrkII, and Rac1. FIG. 3B depicts eIF4 immunoprecipitation (IP) followed by RNA isolation performed to isolate the fraction of the pseudopodia-specific RNA transcripts that undergoes localized translation. The immunoprecipitated RNA was used to perform a real-time PCR array for migration specific genes. The graph shows the existence of Lck, Paxillin, CrkII, and Rac1 (arrows) within the pool of localized transcripts destined for translation in the human GSC pseudopodia.

FIG. 4 shows that phospho-Lck is expressed in human glioblastomas and inhibition of phospho-Lck results in inhibition of paxillin and CrkII activation and loss of pseudopodia formation in hGSCs. FIG. 4A represents an experiment to examine the expression of phospho-Lck in human glioblastomas wherein glioma tissue arrays were stained containing 40 glioblastoma samples (US Biomax, Inc.) with an antibody against pLck-Tyr394 (R&D). 90% of the human glioblastomas tested positive for pLck-Tyr394, while the number of pLck-Tyr394⁺ cells increases with the grading of the tumors. FIGS. 4B and 4C depict hGSCs isolated from two patients with glioblastomas (GSC1 and GSC2) left untreated or treated with 500 nM Lck-I for 2 hours. Lysates were analyzed for phospho(Y394)-Lck, phospho(Y118)-paxillin, phospho(Y221)-CrkII and actin as loading control. Treatment with Lck-I significantly reduced the phosphorylation levels of Lck, paxillin, and CrkII (graph shows densitometric quantification from three independent experiments, error bars represent ±s.d., *:p<0.05). FIG. 4D depicts human GSCs stained for phalloidin-rhodamine and phospho(Y118)-paxillin to identify lamellipodia and active paxillin. Paxillin phosphorylation was reduced in Lck inhibitor-treated cultures as compared with control. FIG. 4E shows human GSCs seeded on a fibronectin substrate that were allowed to attach for 2.5 hours, then left untreated (Control) or treated (Lck-I) with 500 nM Lck-I for 2 hours. Cultures were fixed and stained for phalloidin-rhodamine to identify lamellipodia. FIG. 4F shows the numbers of lamellipodia per cell (30 cells per well, n=4 from three independent experiments, error bars represent ±s.d.). Treatment with Lck inhibitor significantly reduced the number of lamellipodia per cell (:p<0.05). Scale bars represent 200 μm.

FIG. 5 shows inhibition of phospho-Lck results in significant reduction of human glioma cell migration. FIG. 5A depicts lenti-GFP infected human GSCs seeded on DRG axons grown on collagen coated glass coverslips with parallel scratches. Control human GSCs migrate along the parallel axonal bundles forming typical “finger-like” projections (arrows). Treatment of the human GSC-DRG axon co-cultures with Lck-I (Lck inhibitor) results in complete inhibition of migration of human glioma cells along the axonal bundles. FIG. 5B shows human GSCs seeded on purified DRG axon cultures and left untreated (control) or treated with Lck-I for 72 hours (Lck-I). Control human GSCs form GFAP⁺/Ki67⁺ invasive tumor-like structures and exhibit extensive migration of individual glioma cells and integration within the axonal network (inset). Treatment with Lck-I results in inhibition of migration, almost complete lack of integration of tumor cells within the supporting axonal network (inset) and formation of spherical tumor-like structures. FIG. 5C shows quantification of the area of migration of human glioma cells on purified DRG axons. Treatment with Lck-I for 72 hours results in significant reduction of the total area of migration of human glioma cells. FIG. 5D shows the results of quantification performed from 6 independent experiments after normalizing the total area of migration to the interior area of the non-migrating sphere mass. *:P<0.05. FIG. 5E shows human GSCs seeded onto a fibronectin-coated xCELLigence E-plate and allowed to attach for 2 hours. Cell spreading was monitored every 15 seconds following addition of DMSO (CTL) or 500 nM Lck inhibitor. Addition of Lck inhibitor induced an immediate retraction of cell processes as compared with DMSO treated cultures and overall the cell index remained significantly decreased after 10 hours (n=, P<0.05 by Student's t-test, error bars represent ±s.d.).

FIG. 6 shows that inhibition of Lck results in significant reduction of human glioma cell invasion and in vivo administration of Lck-I using an orthotopic xenograft model results in complete inhibition of tumor formation. FIG. 6A depicts human GSCs seeded on D Alvetex scaffolds (Reinnervate) and left untreated or treated with Lck-I for 72 hours. Confocal z-stack images show that inhibition of Lck results in significant inhibition of the invasion properties of human glioma cells. Quantification of the depth of invasion was performed from independent experiments and significance was calculated with unpaired student's t-test (*:p<0.05). FIG. 6B shows inhibition of Lck with continuous infusion of Lck-I in the lateral ventricle for 4 weeks in an orthotopic xenograft model of human glioblastoma results in complete lack of solid tumor formation, absence of tissue necrosis and minimal invasion/integration of human glioma cells within the brain parenchyma. The PBS/DMSO treated animals exhibit all the histopathological characteristics of human glioblastomas with extensive infiltration of tumor cells, tissue necrosis and extensive migration and invasion of tumor cells within the brain parenchyma. Human cells were labeled with anti-human Ki67 and pictures are representative examples of 5 independent experiments.

FIG. 7 shows myelinating DRG-ODC co-cultures in Campenot chambers. DRG neurons were grown in Campenot chambers with pre-made parallel scratches on a collagen substrate. Axons grow in between the scratches forming parallel bundles (left panel). DRG axons grown in parallel orientation were seeded with rat oligodendrocytes and induced to myelinate (right panel). Scale bars represent 200 μm.

FIG. 8 shows that Human glioblastomas express phospho-Lck (S158) and inhibition of Lck with Lck-I is highly specific. FIG. 8A shows staining of a human GBM tissue array containing 40 GBMs with an antibody against phospho-Lck (S158) shows that 90% of GBMs express phosphorylated Lck. FIG. 8B shows that treatment of human glioma cells with 500 nm Lck-I does not affect the phosphorylation state of Src, Fyn, Lyn and Yes, suggesting that the Lck-I is highly specific.

FIG. 9 shows that human glioma stem cells express the stem cell markers CD133 and nestin and can differentiate into astrocytes, oligodendrocytes and neurons. Glioma stem cells (GSCs) isolated from patients with primary GBM and cultured in Neurobasal-A media supplemented with bFGF, EGF and heparin express the stem cell markers Nestin and CD133. Addition of 10% FBS and removal of bFGF, EGF and heparin for 7 days induces differentiation of GSCs into GFAP+ astrocytes, A2B5+ oligodendrocytes and NeuN+ neurons.

### DETAILED DESCRIPTION

Glioblastoma constitutes the most aggressive human cancer due to the extensive migration properties of glioma stem cells (GSCs). The understanding of the molecular mechanisms regulating GSC migration is limited due to the lack of efficient migration models and the inability to study in real time GSC interaction with axons and glial cells. Described herein are transformative methods to perform such studies. These methods include an ex vivo co-culture system that can be used to identify novel mechanisms of GSC migration, which represents a unique platform for testing novel therapeutics on migrating GSCs and related disorders such as glioblastoma. In this context, it was demonstrated that GSCs interact with axons through signaling pathways regulated by local translation of the Src family kinase lymphocyte-specific protein tyrosine kinase (Lck) in GSC pseudopodia. In addition, described herein are methods directed to targeting and inhibiting Lck, which inhibits glioma cell migration and tumor formation in vivo. Also described herein is a novel therapeutic approach for treating glioblastomas through the inhibition of Src family kinases, such as Lck.

Described herein is a transformative approach to study human GSC migration on myelinated and non-myelinated axons. Previously, Applicants developed Dorsal Root Ganglion (DRG) axon Schwann cell co-cultures (Adilakshmi et al., 2011; Rambukkana et al., 2002; Tapinos et al., 2006, each of which is incorporated by reference herein in its entirety). However, there remained a need for culture systems that allow the study of migrating glioma cells, including GSCs.

Applicants have developed an ex vivo system containing DRG axon-oligodendrocyte co-cultures and human GSCs. Through the system described herein, Applicants showed in real-time that GSCs interact with axonal tracks and migrate along the myelinated axons resembling the migration of glioma cells through corpus callosum. In addition, Applicants observed that GSCs interact with neighboring axons through extensive formation of pseudopodia.

Using a previously described Boyden chamber system (Mili et al., 2008, which is incorporated by reference herein in its entirety), Applicants isolated the human GSC pseudopodia and performed RNA immunoprecipitations (IP) with eukaryotic initiation factor 4 (eIF4) to detect local transcripts that may regulate pseudopodia formation and the interaction of GSCs with axons. Applicants discovered local translation of an Lck-regulated signaling pathway comprising Paxillin, Crk-II and Rac1. Due to the recent showing (Ness et al., 2013) that a similar Lck-regulated pathway controls pseudopodia formation in Schwann cells, there is now evidence that this role of Lck is conserved in peripheral and central glia.

Applicants showed, using a glioblastoma tissue array, that activated Lck is expressed in 90% of human glioblastomas and specific inhibition of Lck activity blocks activation of paxillin, Crk-II, the formation of pseudopodia, and the in vitro migration of human GSCs. Applicants also demonstrated that in vivo inhibition of Lck using an orthotopic xenograft mouse model results in significant reduction of human glioma cell migration and inhibition of tumor formation. Using the presently-described novel ex vivo culture system, Applicants analyzed the role of Lck signaling pathways in human GSC migration. Provided herein are methods of regulating Lck as a novel therapeutic target for human glioblastomas.

Glioblastomas are invariably fatal due to the high rate of cancer cell migration and invasion into the surrounding brain parenchyma (Merzak and Pilkington, 1997, which is incorporated by reference herein in its entirety). Current treatment of glioblastomas includes surgical resection of the tumor mass followed by radiation in the vicinity of the resection cavity (Stupp et al., 2005, which is incorporated by reference herein in its entirety) and administration of temozolomide (Stupp and Weber, 2005, which is incorporated by reference herein in its entirety). Even with this multi-therapeutic approach, tumor recurrence is inevitable. This is due to the migration properties of the tumor cells, which invade the brain parenchyma and create multiple “finger like” projections within the brain (Friedl and Wolf, 2003, which is incorporated by reference herein in its entirety) that makes their elimination impossible.

It has long been noted that glioma cells migrate preferentially in association with myelinated tracks (Giese et al., 1996, which is incorporated by reference herein in its entirety) in the brain. However, until now it was not possible to study the real-time interaction of human glioma cells with myelinated or non-myelinated tracks due to lack of efficient in vitro or ex vivo models. Described herein is an ex vivo co-culture system resolves this issue by enabling live imaging of human GSCs during their interaction with myelinated or non-myelinated axons.

Applicants demonstrate that glioma cells form extended pseudopodia to explore the surrounding microenvironment, interact with axons, and migrate. Local exploration of the surrounding environment depends on the ability of the cell to interpret extracellular cues and adopt gene expression and protein synthesis in response to these signals. Positioning the relevant mRNA transcripts at the appropriate place within a cell enables an accelerated response to signaling inputs. With mRNAs concentrated at distinct locations, there is little time spent moving proteins through large regions of cytoplasm (Jung et al., 2014, which is incorporated by reference herein in its entirety).

The particular mRNAs that are selected for translation determines how a cell will react to extracellular signals. A well-described example is the response of the growth cone to attractive or repulsive cues, which is dictated by the local translation of specific mRNAs (Leung et al., 2006; Piper et al., 2006; Wu et al., 2005; Yao et al., 2006, each of which is incorporated by reference herein in its entirety). Such stimulus-driven mRNA-specific local translation spatiotemporally links signal reception to gene function (Jung et al., 2014, which is incorporated by reference herein in its entirety) and is particularly relevant to the regulation of cancer cell migration and invasion.

Applicants discovered that as glioma cells interact with surrounding axons and glia, they extend and retract numerous pseudopodia to explore the environment and migrate along myelinated axon tracks. This is a dynamic process that requires acute response to local signals through the on-site translation of subcellularly-localized mRNAs in the pseudopodia. The role of an Lck-regulated pathway for lamellipodia formation and migration of Schwann cells (Ness et al., 2013) was recently described. In the present application, Applicants show spatial localization of mRNAs for the Lck pathway in pseudopodia of human glioma cells. All members of the Lck-regulated pathway undergo eIF4-dependent localized translation in pseudopodia, demonstrating a central role of this pathway in the dynamic regulation of cytoskeletal rearrangements of human glioma cells during exploration of their microenvironment and subsequently in control of their migration. In the present application, Applicants demonstrate that phospho-Lck is present in 90% of the 40 glioblastomas tested, which suggests a significant role of Lck in human glioblastomas.

Recently, it was proposed that Lck mediates the expansion of the CD133+GSC pool following ionizing radiation of glioblastomas and that inhibition of Lck inhibits the radiation induced expression of CD133, Nestin and Musashi in GSCs (Kim et al., 2010, which is incorporated by reference herein in its entirety). In the present application, Applicants demonstrate that inhibition of Lck activity results in significant inhibition of pseudopodia formation, blocking of the activation of Paxillin and CrkII, and significant decrease in the migration of human GSCs.

As demonstrated here, in vivo administration of an Lck inhibitor (Lck-I) in an orthotopic xenograft model of human glioblastomas shows significant inhibition of tumor migration and invasion. The inventors have now demonstrated that Lck is an attractive target for development of novel therapies for human glioblastoma. Accordingly, described herein are methods directed to targeting Lck in the treatment of human glioblastoma. Also described herein is a method for identifying inhibitors of glioma cell migration, including inhibitors of Lck and/or other Src kinases.

Recently, the focus for development of new therapies for glioblastomas has shifted towards molecular therapies targeting individual proteins or signaling pathways that control glioma cell migration and invasion (Demuth and Berens, 2004, which is incorporated by reference herein in its entirety). In this respect EMD121974 (Cilengitide), a cyclic peptide targeting the RGD-motif of integrins, was introduced. EMD121974 is designed to act in an anti-angiogenic manner, blocking avb- and avb5-integrin mediated interaction between endothelial cells and extracellular matrix (ECM), which appears to be crucial for angiogenesis (Hynes, 2007, which is incorporated by reference herein in its entirety). By targeting two integrins, which have also been linked to glioma invasion (Tonn et al., 1998, which is incorporated by reference herein in its entirety), this compound may also exhibit anti-invasive potential in human gliomas. It has been shown that EMD121974 induces apoptosis in U87 glioma cells cultured on tenascin and vitronectin (Taga et al., 2002, which is incorporated by reference herein in its entirety) and significant regression of glioma xenografts (MacDonald et al., 2001, which is incorporated by reference herein in its entirety).

In addition to targeting integrins, targeting Src kinases with Dasatinib is currently under various clinical trials for the treatment of human glioblastoma (Lassman et al., 2015, which is incorporated by reference herein in its entirety). Dasatinib is an oral multi-BCR/AbI and Src family tyrosine kinase inhibitor approved for first line use in patients with chronic myelogenous leukemia and Philadelphia chromosome-positive acute lymphoblastic leukemia (Han et al., 2014, which is incorporated by reference herein in its entirety).

The present inventors have shown that inhibiting Src kinases, such as Lck, offer several advantages, including the following three, compared to the current trials. First, Lck is a downstream effector of the integrin signaling pathway and therefore targeting Lck is more specific in respect to the cytoskeletal changes that control glioma cell migration. Second, as demonstrated herein, phospho-Lck is expressed preferentially in glioma cells, which makes it far more selective as a target comparing to integrins that are expressed in endothelial cells as well. Third, targeting Lck is a superior alternative to Dasatinib, which is a pan-inhibitor and affects EGF and VEGF expression, making it more likely to have severe side effects in patients with brain tumors.

Methods described herein are directed to inhibiting or reducing the expression of a Src family kinase, such as Lck, gene, gene product, and/or protein. These methods reduce or inhibit the expression of the Src family kinase gene, gene product, and/or protein for treating glioblastoma in a subject (e.g., a human), inhibiting the migration of a glial cell in a subject or in culture, and/or reducing or inhibiting tumor formation in a subject (e.g., a human). In some embodiments, the expression of the Src family kinase gene, gene product, and/or protein is inhibited or reduced in a glial cell in culture or in a subject. In certain embodiments, the expression of the Src family kinase gene, gene product, and/or protein is inhibited or reduced in a gliomal cell of a subject, including humans.

In certain embodiments, the expression of the Src family kinase gene, gene product, and/or protein is reduced or inhibited by a method comprising administering an antisense oligonucleotide to the cell and/or subject. In general, an antisense oligonucleotide includes a single stranded oligonucleotide that is complementary to a messenger RNA (mRNA) strand transcribed within a cell. An antisense oligonucleotide can reduce or inhibit the expression of the target mRNA by base-pairing with the target mRNA and obstructing its translation into a protein product. In certain embodiments, the antisense oligonucleotide targets a transcript that encodes the Src family kinase protein and/or is encoded by the Src family kinase gene. In some embodiments, the antisense oligonucleotide is an RNA molecule.

In some embodiments, the expression of the Src family kinase gene, gene product, and/or protein is reduced or inhibited by a method comprising RNA interference (RNAi). Generally, RNAi is a process in which RNA molecules inhibit expression of a gene or protein by either causing the destruction of targeted mRNA molecules or preventing or obstructing the translation of the targeted mRNAs. Two types of effector RNAs that are known to be used for reducing gene or protein expression via the RNAi pathway are small interfering RNA (siRNA) and small hairpin RNA (shRNA).

siRNAs are a small class of double-stranded RNA molecules that have complementary sequences with targeted polynucleotides (e.g., mRNA). In general, it is thought that siRNAs function by inducing the destruction of targeted mRNAs after transcription, thereby preventing translation of the targeted mRNA. In certain embodiments described herein, the expression of the Src family kinase gene, gene product, and/or protein is reduced or inhibited by a method comprising administering an siRNA to the cell and/or subject. In some embodiments, the siRNA molecule targets a transcript (e.g., mRNA) that encodes the Src family kinase protein and/or is encoded by the Src family kinase gene.

Generally, shRNAs are artificial RNA molecules (e.g., synthetically produced) that contain a hairpin structure and can be used to silence gene expression through complementarity (hybridization) to target polynucleotides (e.g., mRNA). It is thought that perfect complementarity leads to mRNA cleavage whereas imperfect complementary results in translational repression of the target mRNA. In certain embodiments, the expression of the Src family kinase gene, gene product, and/or protein is reduced or inhibited by a method comprising administering a shRNA to the cell and/or subject. In some embodiments, the shRNA molecule targets a transcript (e.g., mRNA) that encodes the Src family kinase protein and/or is encoded by the Src family kinase gene.

The expression of the Src family kinase gene, gene product, and/or protein is reduced or inhibited by a method, provided herein, comprising gene editing. Gene editing can be accomplished by any one of several well-known techniques in the art, including, but not limited to, the use of nucleases. Examples of nucleases that can be used for gene editing include zinc finger nucleases (ZFNs), transcription Activator-Like Effector Nucleases (TALENs), the Clustered Regularly Interspaced Short Palindromic Repeats (CRISPR)/CRISPR-associated (Cas) (CRISPR/Cas) system, and engineered meganucleases. In certain embodiments, the expression of the Src family kinase gene, gene product, and/or protein is reduced or inhibited by a method comprising administering a CRISPR/Cas system to a cell and/or subject. In some embodiments, the CRISPR/Cas system targets a Src family kinase gene, including, but not limited to, Lck.

Thus, provided herein is a transformative method to study the interaction of human glioma cells with myelinated and non-myelinated axons. Applicants' ex vivo co-culture system serves as a platform for the identification of new pathways regulating glioma cell migration and the development of novel therapies. In this context, Applicants discovered that spatial localization and translation of Lck mRNA in glioma cell pseudopodia regulates a local pathway that controls human glioma cell migration. Targeted inhibition of Lck constitutes a novel therapy for human glioblastomas.

In summary, migration of tumor propagating glioma stem cells (GSCs) within the brain parenchyma makes glioblastoma one of the most aggressive and lethal human tumors. Studies of the cellular and molecular mechanisms underlying human GSC migration were hindered by the lack of efficient migration models. Applicants developed a DRG axon-oligodendrocyte co-culture method to study in real-time the migration and interaction of GSCs with axons, which occurs through the extensive formation of pseudopodia. Isolation of pseudopodia-localized RNA followed by eIF4 RNA-IP revealed local transcripts of Lck, Paxillin, Crk-II, and Rac1 that undergo eIF4-dependent translation. Inhibition of Lck blocks the activation of this pathway, the formation of pseudopodia, and the migration of GSCs. In vivo administration of a highly-specific Lck inhibitor using an orthotopic xenograft mouse model results in significant inhibition of tumor formation and GSC migration. Targeting this locally translated Lck-dependent pathway constitutes a novel treatment paradigm for human glioblastomas.

Provided herein is a method for treating glioblastoma in a subject comprising administering to the subject an inhibitor of a Src family kinase, wherein the inhibitor is not Dasatinib. In some embodiments, the Src family kinase is lymphocyte-specific protein tyrosine kinase (Lck). In certain embodiments, the inhibitor is a small molecule. In some embodiments, the inhibitor reduces the level of phosphorylation of the Src family kinase. In certain embodiments, the inhibitor reduces the level of phosphorylation of paxillin. In some embodiments, the inhibitor is A770041. In some embodiments, the subject is a human, mouse, rat, or dog.

Also provided herein is a method for inhibiting the migration of a glial cell comprising contacting the glial cell with an inhibitor of a Src family kinase, wherein the inhibitor is not Dasatinib. In some embodiments, the glial cell is a glioma stem cell (GSC). In some embodiments, the number of pseudopodia formed in the glial cell contacted with the inhibitor is reduced compared to the number of pseudopodia formed in a migrating glial cell not contacted with the inhibitor. In certain embodiments, the Src family kinase is lymphocyte-specific protein tyrosine kinase (Lck). In some embodiments, the inhibitor is a small molecule. In certain embodiments, the inhibitor reduces the level of phosphorylation of the Src family kinase. In some embodiments, the inhibitor reduces the level of phosphorylation of paxillin. In certain embodiments, the inhibitor is A770041.

Also provided herein is a method for reducing or inhibiting tumor formation in a subject comprising administering to the subject an inhibitor of a Src family kinase, wherein the inhibitor is not Dasatinib. In some embodiments, the tumor is a glioblastoma. In certain embodiments, the Src family kinase is lymphocyte-specific protein tyrosine kinase (Lck). In some embodiments, the inhibitor is a small molecule. In certain embodiments, the inhibitor reduces the level of phosphorylation of the Src family kinase. In certain embodiments, inhibitor reduces the level of phosphorylation of paxillin. In some embodiments, the inhibitor is A770041. In certain embodiments, the subject is a mammal. In some embodiments, the mammal is a dog, mouse, rat, or human.

Also provided herein is an ex vivo co-culture system comprising a Dorsal Root Ganglion (DRG) axon-oligodendrocyte and a gliomal cell. In some embodiments, the gliomal cell is a glioma stem cell (GSC). In certain embodiments, the DRG axon-oligodendrocyte is myelinated. In some embodiments, the gliomal cell expresses green fluorescent protein (GFP). In certain embodiments, the GFP is introduced into the gliomal cell via viral infection. In some embodiments, the viral infection comprises a Lentivirus.

Also provided herein is a method for identifying an inhibitor of glial cell migration or movement comprising contacting a glial cell in an ex vivo co-culture system with a putative glial cell migration or movement inhibitor; imaging the migration or movement of the glial cell; and comparing the migration or movement of the glial cell contacted with the putative inhibitor to the migration or movement of a glial cell in the co-culture system that has not been contacted with the putative inhibitor, wherein a putative inhibitor that reduces or prevents migration or movement of the glial cell is characterized as an inhibitor of glial cell migration or movement. In some embodiments, the co-culture system comprises a Dorsal Root Ganglion (DRG) axon-oligodendrocyte. In certain embodiments, the cell migration or movement of the glial cell is measured by quantifying the surface area covered by the glial cell. In some embodiments, the quantification of the surface area comprises detection of electrical impedance. In certain embodiments, the glial cell is a gliomal cell. In some embodiments, the gliomal cell is a glioma stem cell (GSC). Also provided herein is a method for treating glioblastoma in a subject comprising administering to the subject an inhibitor of glial cell migration or movement identified by the method described above.

Also provided herein is a method for imaging the migration or movement of a glial cell comprising live imaging a glial cell present in an ex vivo co-culture system. In some embodiments, the co-culture system comprises a Dorsal Root Ganglion (DRG) axon-oligodendrocyte. In certain embodiments, the live imaging comprises time-lapse photography. In some embodiments, the glial cell is a gliomal cell. In certain embodiments, the gliomal cell is a glioma stem cell (GSC).

Also provided herein is a method for isolating pseudopodia of a gliomal cell, the method comprising seeding the gliomal cell on a well comprising pores and fibronectin; culturing the gliomal cell in the well; and isolating pseudopodia of the gliomal cell from the sides of the well. In some embodiments, the pores are at least about 1 μm. In certain embodiments, the pores are about 1 μm. In some embodiments, the fibronectin is human fibronectin. In certain embodiments, the pseudopodia are scraped from the sides of the well. In some embodiments, the well comprises a Boyden chamber. In certain embodiments, the gliomal cell is a glioma stem cell (GSC).

Also provided herein is an isolated pseudopodium derived from a gliomal cell. In some embodiments, the gliomal cell is a glioma stem cell (GSC). In certain embodiments, the pseudopodia do not express cytoplasmic marker Grp75. In some embodiments, the pseudopodia are isolated by a method comprising seeding the gliomal cell on a well comprising pores and fibronectin; culturing the gliomal cell in the well; and isolating pseudopodia derived from the gliomal cell from the sides of the well.

Also provided herein is a method of identifying RNA present in the pseudopodia of a gliomal cell, the method comprising isolating the pseudopodia of the gliomal cell per the method of any one of claims 43-49 and isolating RNA from the isolated pseudopodia. In some embodiments, at least a portion of the isolated RNA is characterized by a process comprising real-time polymerase chain reaction (PCR). In certain embodiments, at least a portion of the RNA is characterized by a process comprising RNA immunoprecipitation. In some embodiments, the RNA immunoprecipitation is performed on a eukaryotic initiation factor 4 (eIF4) protein.

Also provided herein is a method for treating glioblastoma in a subject comprising reducing the expression of a Src family kinase protein in a gliomal cell of the subject. Also provided herein a method for inhibiting the migration of a glial cell comprising reducing the expression of a Src family kinase protein in the glial cell. Also provided herein is a method for reducing or inhibiting tumor formation in a subject comprising reducing the expression of a Src family kinase protein in a gliomal cell of the subject. In some embodiments, the Src family kinase protein is lymphocyte-specific protein tyrosine kinase (Lck). In certain embodiments, the expression of the Src family kinase protein is reduced by a method comprising administering an antisense oligonucleotide to the gliomal cell of the subject. In some embodiments, the antisense oligonucleotide targets a transcript encoding the Src family kinase protein. In certain embodiments, the expression of the Src family kinase protein is reduced by a method comprising RNA interference in the gliomal cell of the subject or the glial cell. In some embodiments, the RNA interference method comprises administering a small interfering RNA (siRNA) to the gliomal cell of the subject or the glial cell. In certain embodiments, the siRNA targets a transcript encoding the Src family kinase protein. In some embodiments, the RNA interference method comprises administering a small hairpin RNA (shRNA) to the gliomal cell of the subject or the glial cell. In certain embodiments, the shRNA targets a transcript encoding the Src family kinase protein. In some embodiments, the expression of the Src family kinase protein is reduced by a method comprising gene editing. In certain embodiments, the gene editing method comprises altering the expression of a gene that encodes the Src family kinase protein. In some embodiments, the gene editing method comprises administering a clustered regularly interspaced short palindromic repeats (CRISPR)/CRISPR-associated (Cas) (CRISPR/Cas) system to the gliomal cell of the subject or the glial cell.

### EXAMPLES

### Example 1—Development of Ex Vivo Co-Culture System to Study Interaction of Human GSCs with Axons

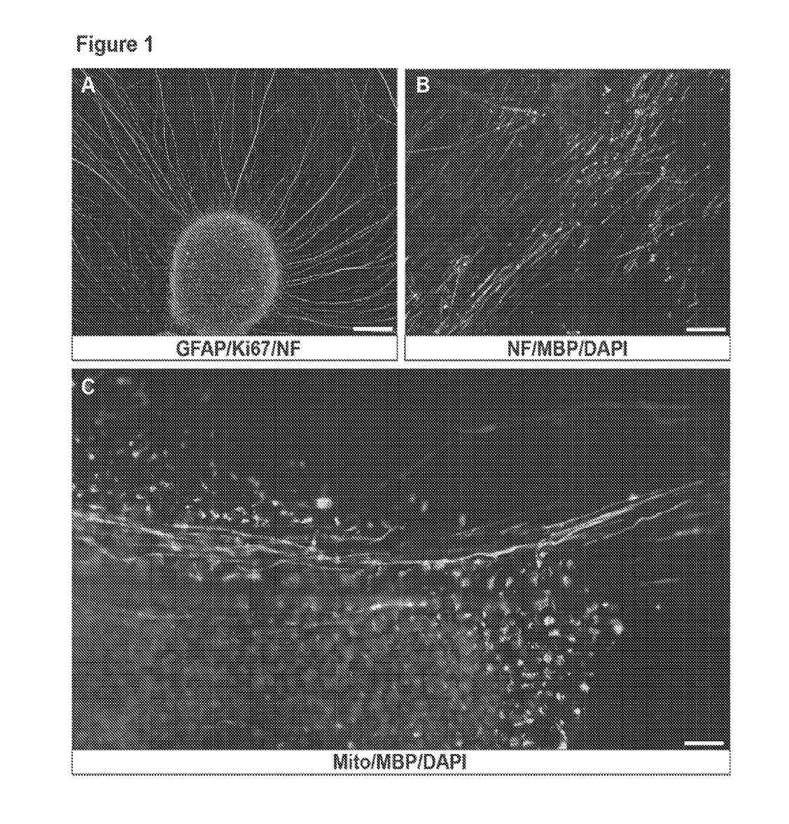

Human GSCs were isolated from glioblastoma tumor tissue as shown before (Galli et al., 2004) following surgical resection from patients at Geisinger Clinic. The GSCs were cultured as primary, secondary, and tertiary neurospheres to verify self-renewal and stained positive for the stem cell markers nestin and CD133 (FIG. 9). All GSCs are capable of differentiating to GFAP+ astrocytes, A2B5+ oligodendrocytes and NeuN+ neurons (FIG. 9). In order to study the interaction of human GSCs with axons Applicants generated purified DRG axons as previously described (Rambukkana et al., 2002; Tapinos et al., 2006; Windebank et al., 1985; Wood, 1976). Next, Applicants seeded the purified DRG axons with human GSCs, which they integrate within the axonal network forming GFAP+/Ki67+ tumor-like structures (FIG. 1A) and exhibit migration of GSCs in between the axons (FIG. 1B). To determine how human GSCs interact with myelinated axons, Applicants seeded DRG axon cultures with purified rat oligodendrocytes and induced myelination as previously described (Chan et al., 2004; Dugas et al., 2006; Ruffini et al., 2004) (FIG. 1 C). Addition of GSCs on the myelinated DRG-oligodendrocyte co-cultures showed that GSCs preferentially migrate in association with myelinated axons (FIG. 1D).

### Example 2—Human GSCs Interact with Axons Through the Formation of Pseudopodia

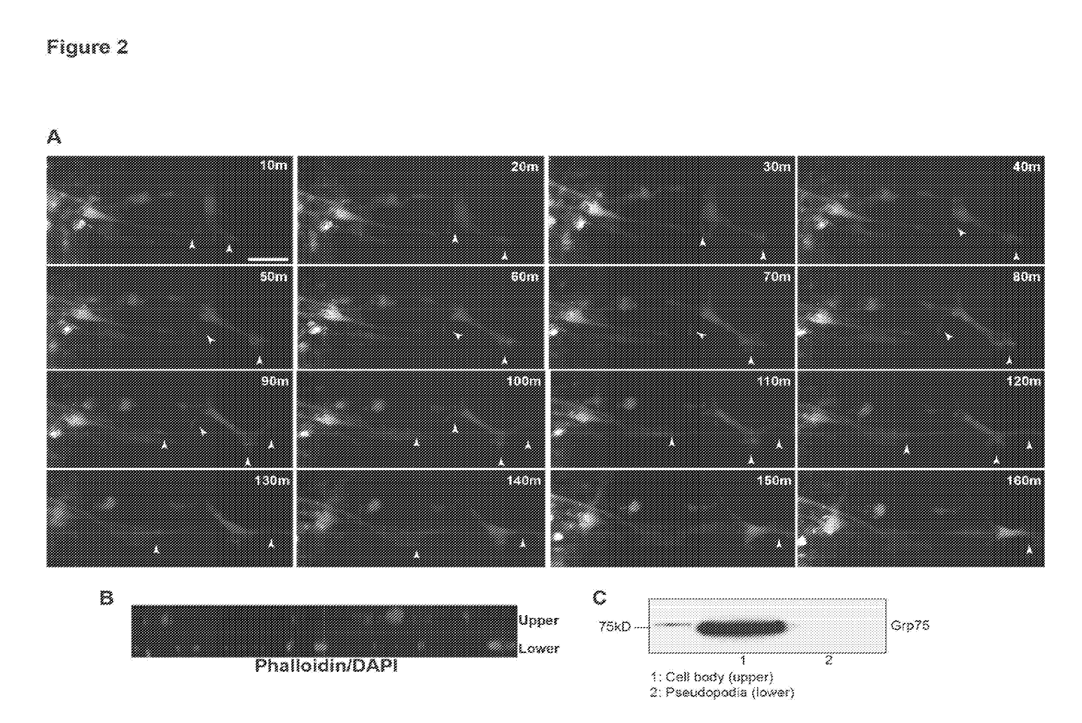

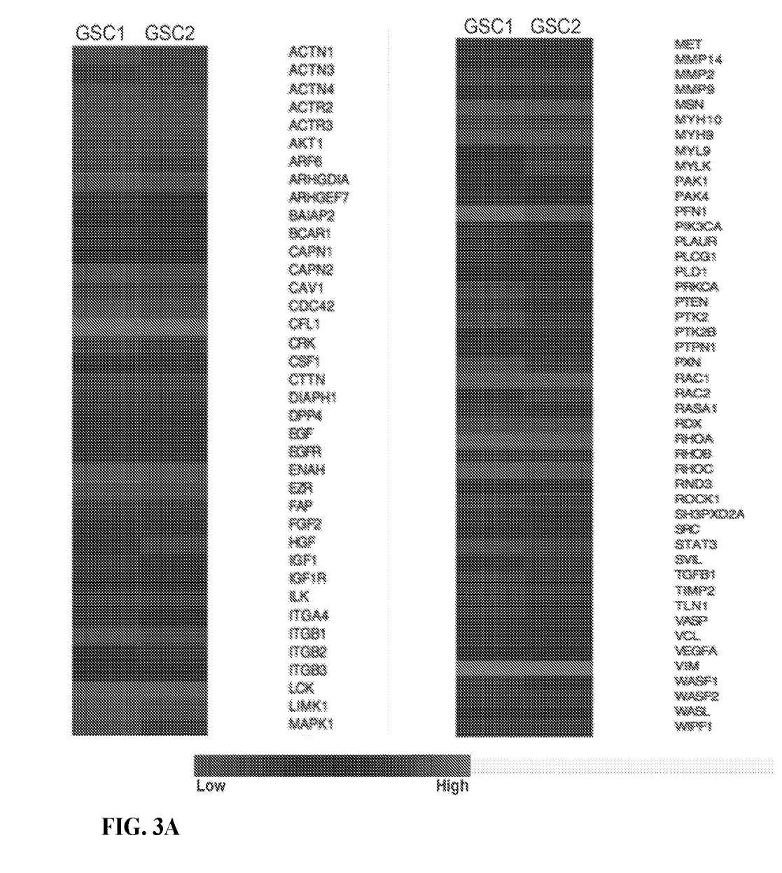

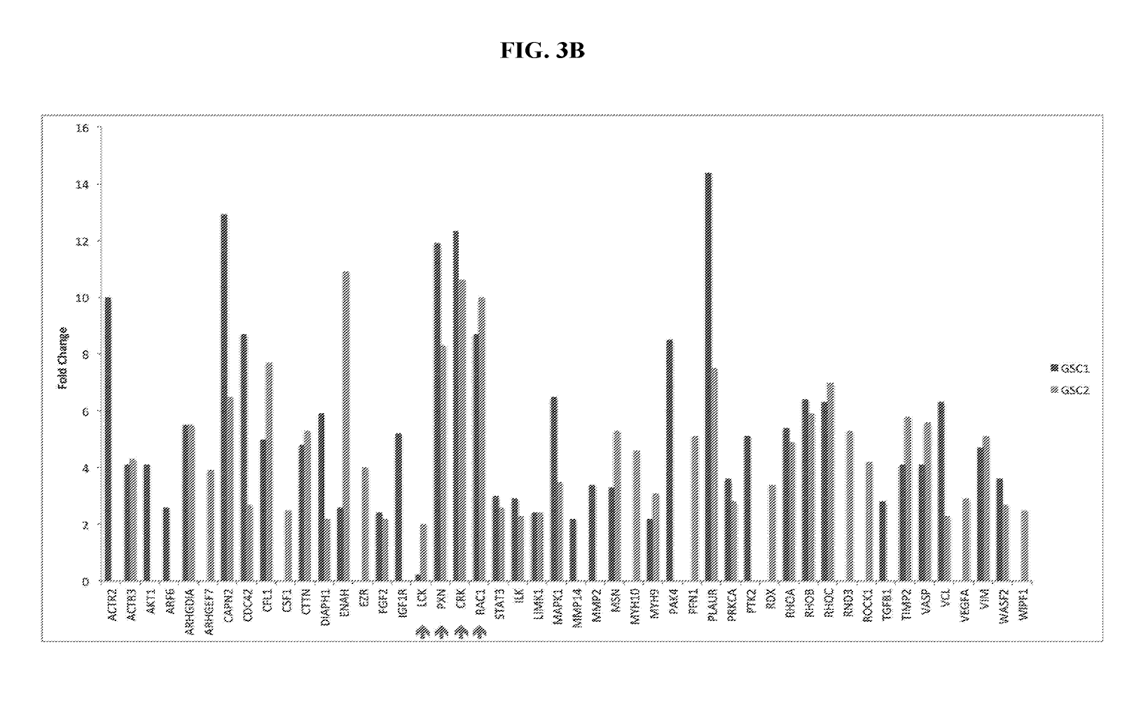

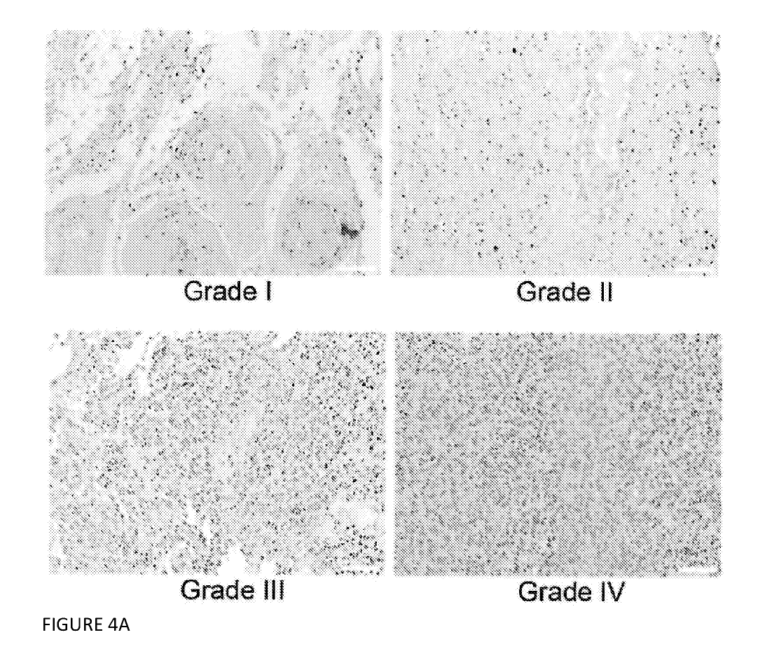

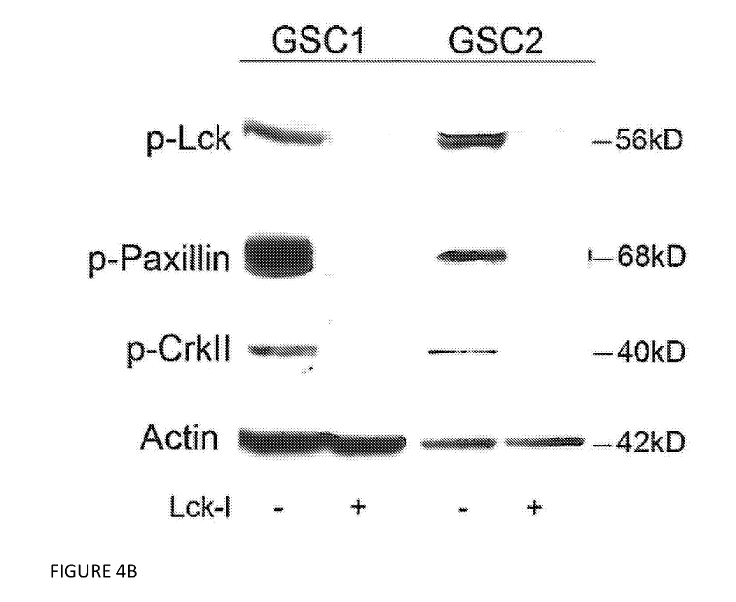

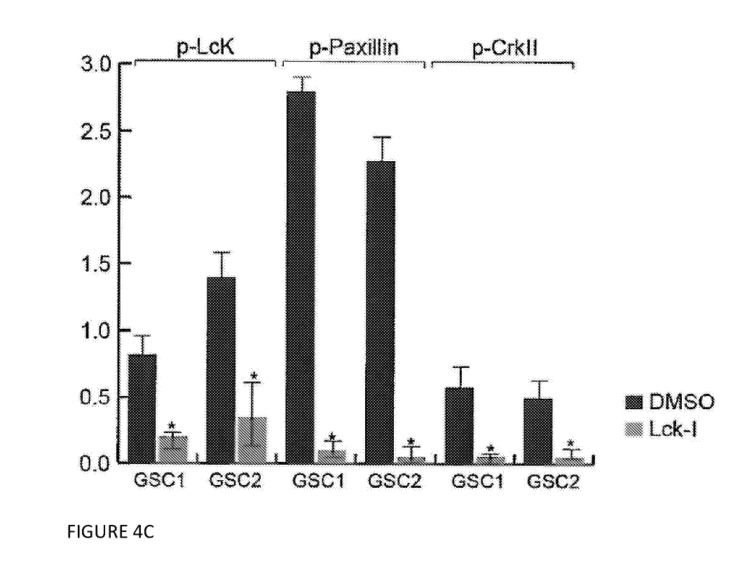

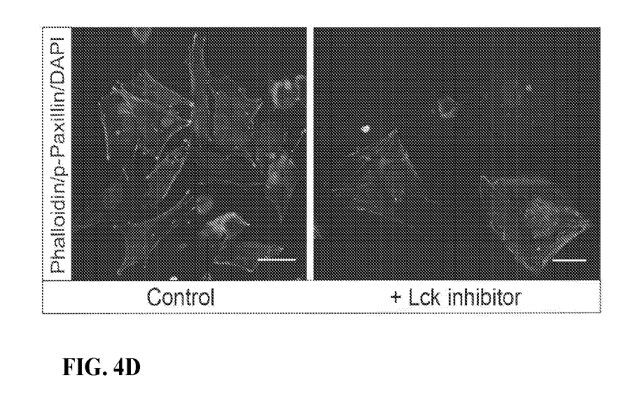

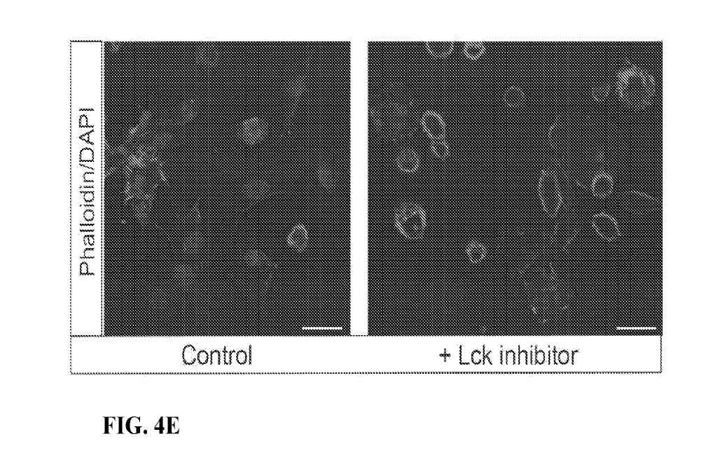

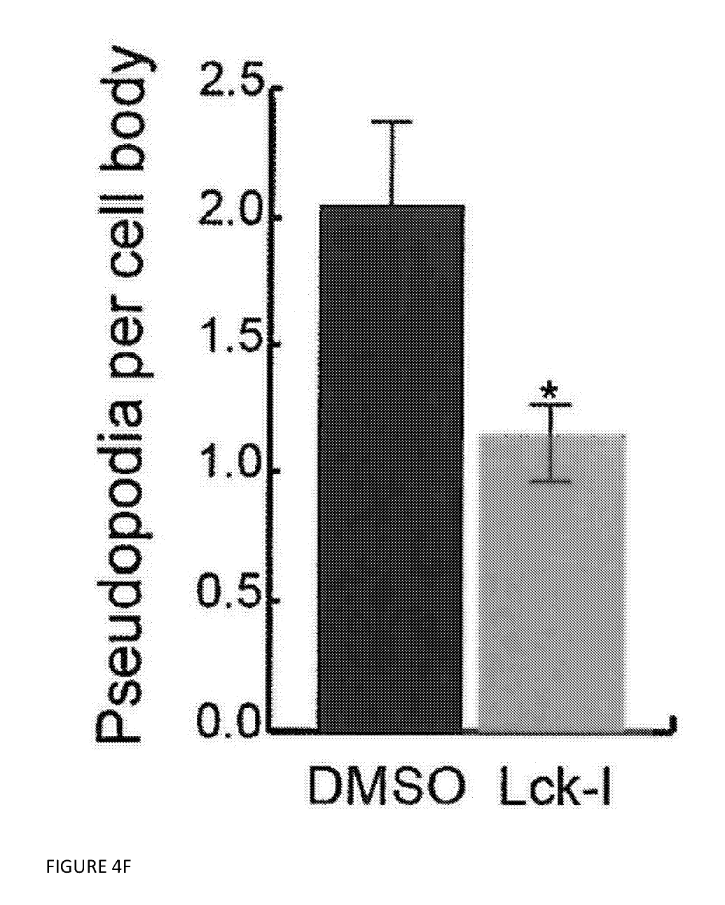

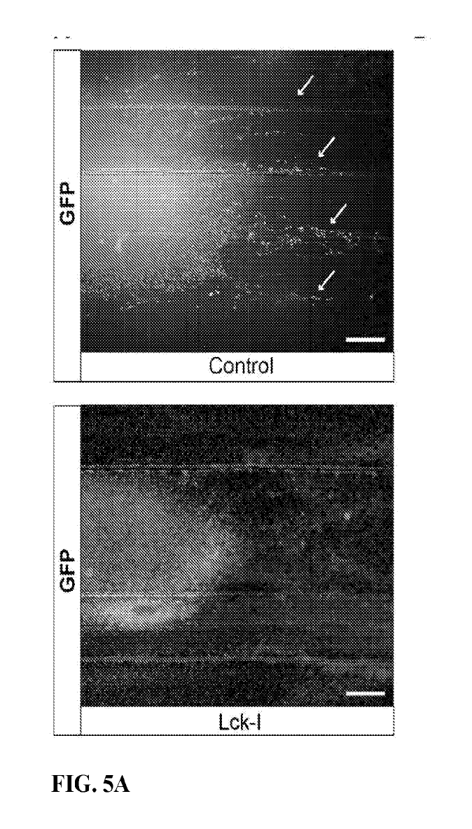

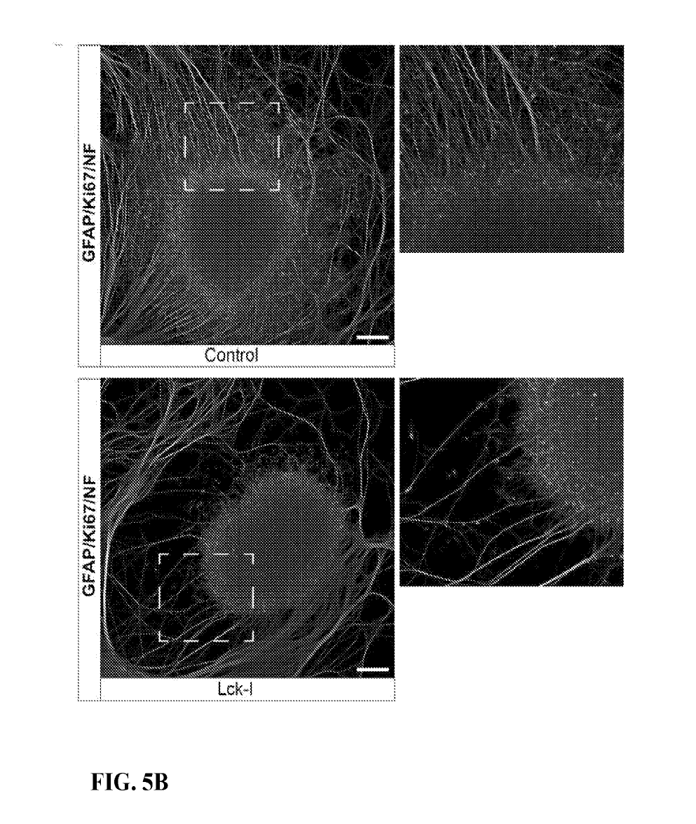

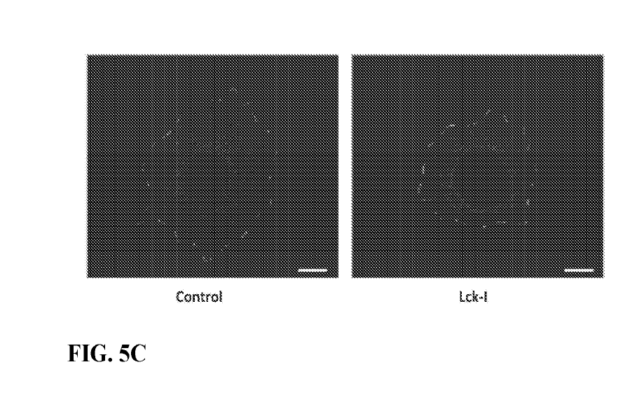

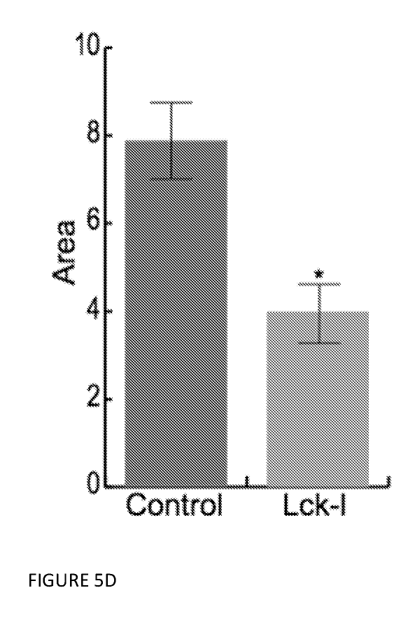

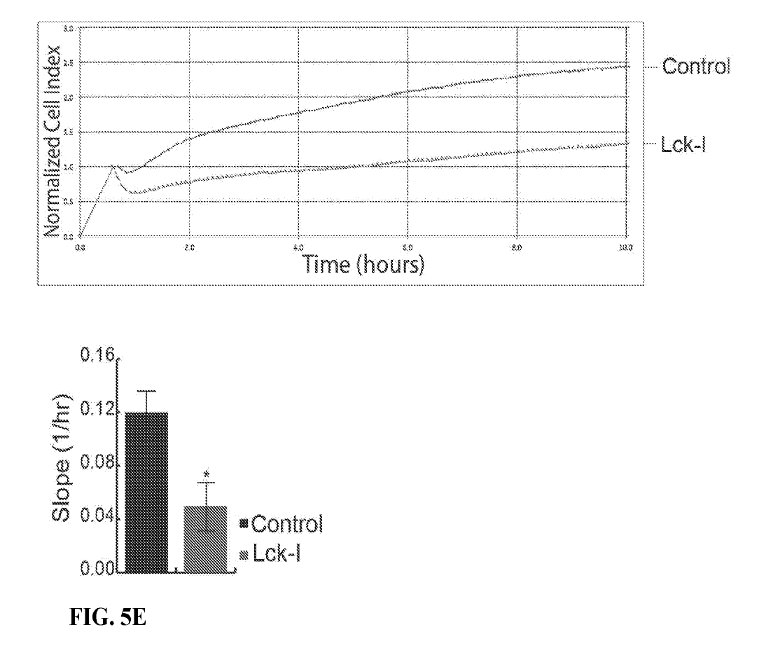

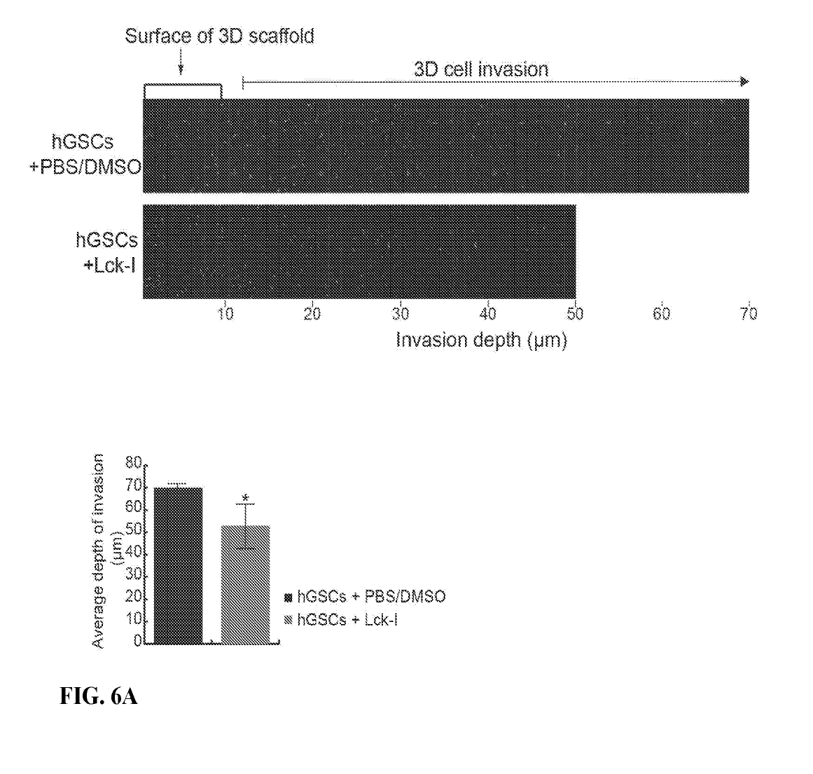

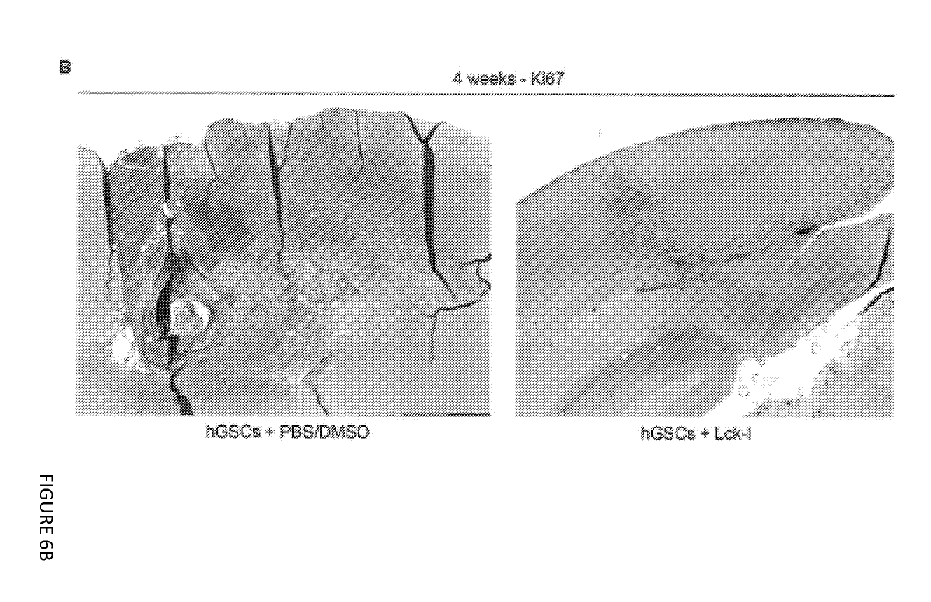

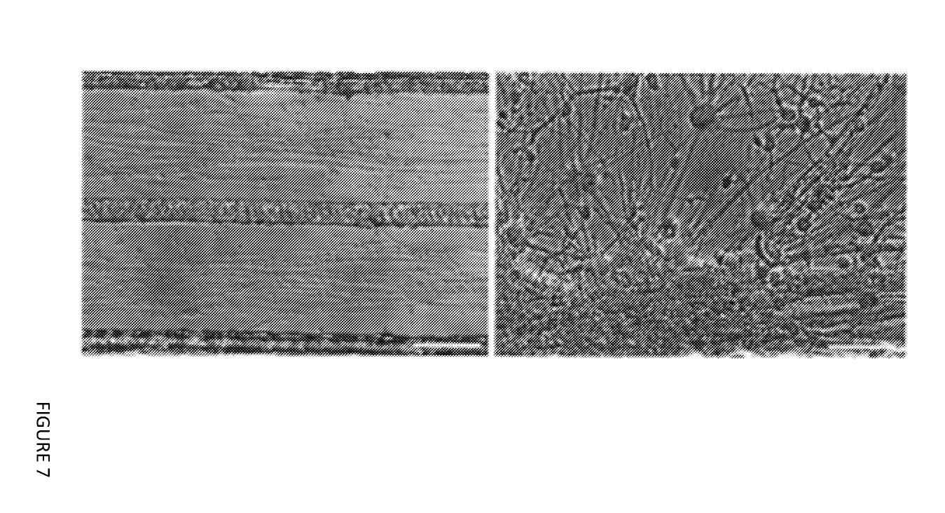

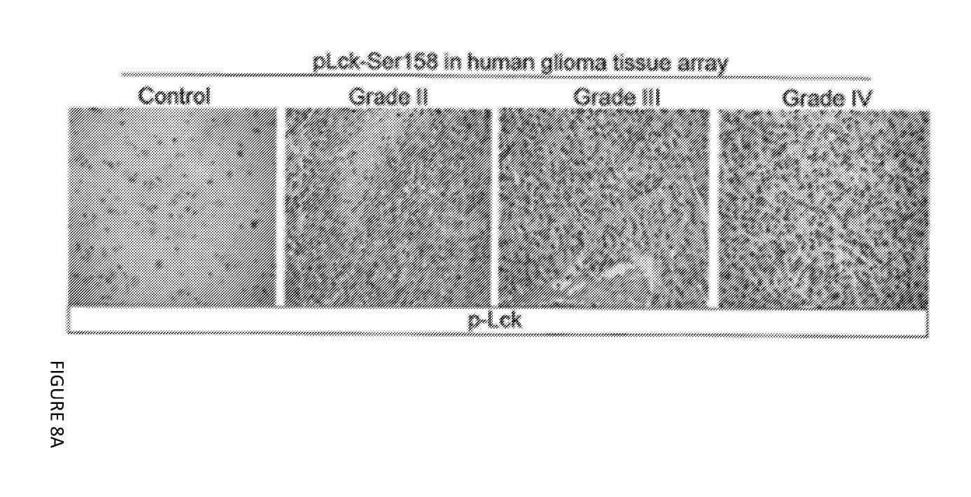

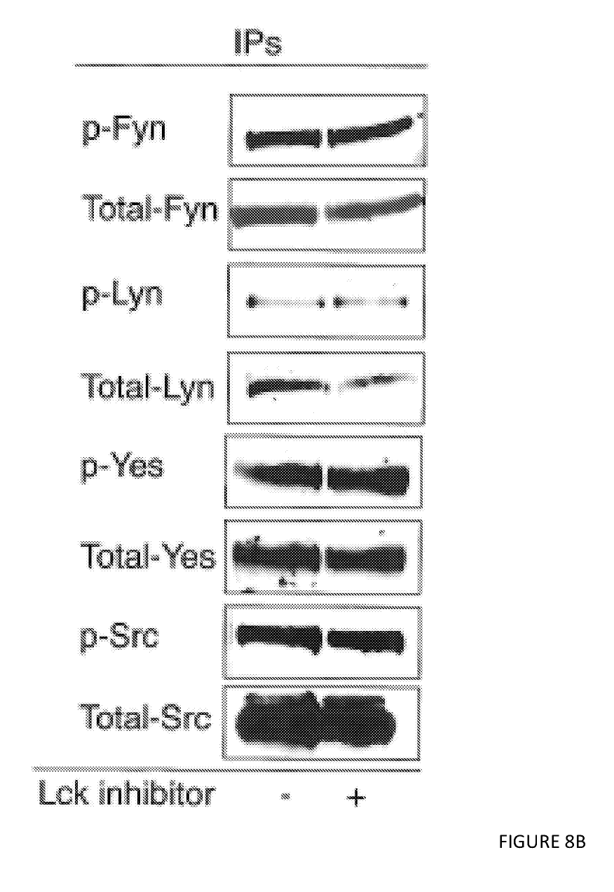

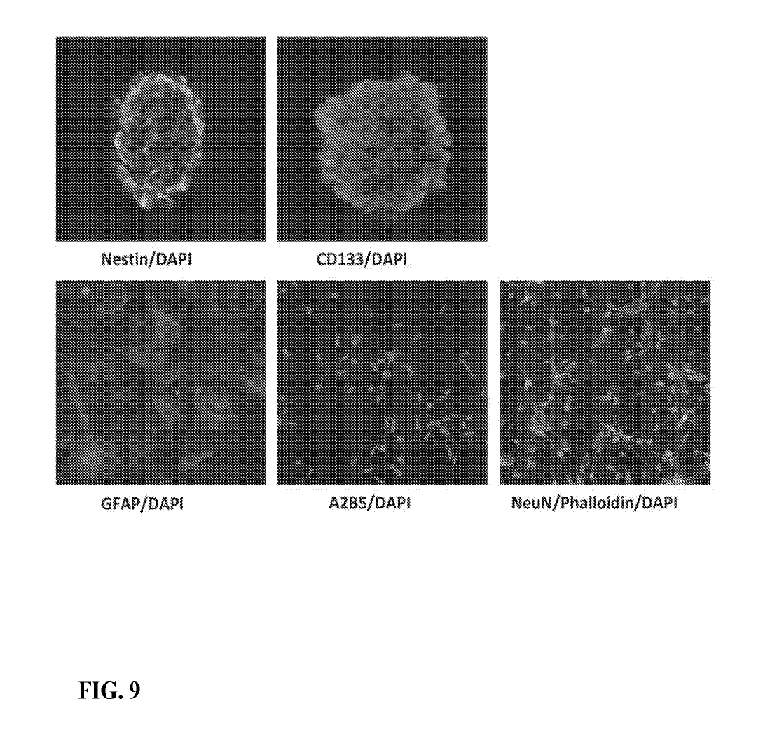

To study the interaction of GSCs with non-myelinated axons in real time, Applicants infected human GSCs with a Lentivirus expressing GFP, seeded the Lenti-GFP+ GSCs on purified DRG axons, and performed live imaging for 5 days. GSCs interact with and invade the axonal network forming extensive pseudopodia that associate with axons. In several instances human GSCs “pull” individual axons and separate axon bundles with their pseudopodia (FIG. 2A). To determine how GSCs interact with myelinated axons, Applicants prepared DRG-oligodendrocyte myelinated axonal tracks in parallel orientation using Campenot chambers on laminin substrate with parallel scratches for guidance (FIG. 14). Time-lapse photography of GSCs on parallel myelinated axons shows the active formation and retraction of multiple pseudopodia as the GSCs interact with the myelinated fibers (FIG. 2A). To isolate the GSC pseudopodia, Applicants used a Boyden chamber separated by a membrane with 1μ pore openings as previously described (Cho and Klemke, 2002; Mili et al., 2008; Wang et al., 2007; Wang and Klemke, 2007). Confocal z-stack images of the chambers shows that only the GSC pseudopodia cross the 1μ pore while the nuclei and the rest of the cell body remain on the upper chamber (FIG. 2B). Protein isolation from the upper and lower chambers shows that the pseudopodia fraction is pure and does not express the cytoplasmic marker Grp75 (FIG. 2C).

### Example —GSC Pseudopodia Contain Migration-Specific RNA Transcripts

To determine if the human GSC pseudopodia contain transcripts that encode proteins with a role in cellular migration, Applicants isolated RNA from the purified pseudopodia to perform a real time PCR array for migration specific genes (SA Biosciences). Applicants used RNA from GSCs of two patients representing mesenchymal and classical subtypes of glioblastomas according to the TCGA classification (Verhaak et al., 2010). Applicants demonstrated that RNA isolated from pseudopodia of human GSCs contains various migration specific transcripts (FIG. 3A). In particular, the associations of cdc42, cofilin-1, ezrin, ILK, LIMK, STAT3, MMP2, and ITGB1 with glioblastoma cell migration and invasion have been recently shown either independently or as parts of more complex intracellular pathways (de la Iglesia et al., 2009; Koul et al., 2005; Milinkovic et al., 2013; Morales et al., 2010; Park et al., 2014; Reyes et al., 2013; Wick et al., 2001). Applicants recently described the presence of a migration specific pathway in Schwann cells consisting of Lck, Paxillin, CrkII and Rac1 (Ness et al., 2013). Transcripts for all members of this pathway were expressed in all GSC pseudopodia (FIG. 3A), suggesting a conserved role of this Lck-regulated pathway for cellular migration between peripheral (Schwann cells) and central glia.

### Example 4—Transcripts of the Lck-Regulated Pathway Undergo eIF4-Dependent Local Translation in Pseudopodia

To examine if the RNA transcripts localized in GSC pseudopodia undergo active on-site translation, Applicants performed an eIF4 RNA immunoprecipitation using the pseudopodia RNA as template since eIF4 bound transcripts are subject to steady-state translation (Ishigaki et al., 2001). Applicants determined that a significant fraction of the pseudopodia RNA content undergoes eIF4-regulated translation (FIG. 3B). In addition, transcripts of Lck, Paxillin, CrkII, and Rac1 present in GSC pseudopodia are subject to local translation independent of glioblastoma subtype classification (FIG. 3B, arrows). Applicants' data demonstrate that the localization of mRNAs of the Lck regulated pathway in pseudopodia and the subsequent on-site translation is a dedicated mechanism in human GSCs for the efficient response to localized signals for cellular migration.

### Example 5—Phospho-Lck is Expressed in the Majority of Human Glioblastomas and Inhibition of Lck Blocks the Activation of Paxillin and CrkII in GSCs

Lck is regulated by phosphorylation on multiple residues, including Ser-158 in the SH2 domain (Reynolds et al., 1992) and Tyr-394 (Yamaguchi and Hendrickson, 1996). To examine the expression of phospho-Lck in human glioblastomas, Applicants stained glioma tissue arrays containing 40 glioblastoma samples in duplicate (US Biomax, Inc.) with antibodies against pLck-Tyr394 (R&D) and pLck-Ser158 (Abcam). Applicants discovered that 90% of the human glioblastomas tested positive for pLck-Tyr394 (FIG. 4A) and pLck-Ser158 (FIG. 8A). Applicants recently showed that phosphorylation of Lck on Tyr394 results in activation of a downstream pathway consisted of Paxillin, CrkII and Rac-1 in Schwann cells (Ness et al., 2013). To elucidate the role of Lck signalling on paxillin and CrkII in human GSCs, Applicants used an Lck inhibitor (Lck-I) (A770041, Cedarlane) that specifically binds to the Lck active site at nanomolar concentrations and shows 8-, 60- and 300-fold specificity for Lck over Src kinase family members Lyn, Src and Fyn, respectively (Burchat et al., 2006; Stachlewitz et al., 2005). Inhibition of Lck with Lck-I shows no effect on the phosphorylation state of Src, Yes, Lyn and Fyn in human GSCs (FIG. 8B). Human GSCs seeded on fibronectin and treated with Lck-I show a significant reduction in total levels of phospho(Y394)-Lck, phospho(Y118)-paxillin and phospho(Y221)-CrkII (FIGS. 4B & 4C, p<0.005). Paxillin is a primary molecular adaptor protein involved in the activation of multiple downstream signaling pathways, localizes to focal adhesion contacts, and stimulates pseudopodia formation and cytoskeletal rearrangement (Deakin and Turner, 2008). Treatment of human GSCs with the Lck-I results in almost complete absence of phospho-paxillin(Y118) from focal adhesions (FIG. 4D).

### Example 6—Lck Regulates the Formation of Pseudopodia and the Migration and Invasion of Human GSCs

To determine the effect of Lck inhibition on pseudopodia formation, human GSCs were seeded on a fibronectin substrate then treated with Lck-I or dimethylsulphoxide (DMSO) (FIG. 4E). The number of pseudopodia formed by inhibitor-treated cells was significantly lower than in untreated cells (FIG. 4F, p<0.005).

As inhibition of Lck signalling has a dramatic effect on pseudopodia extension and retraction, Applicants investigated whether Lck may also function to regulate the migration and invasion of human GSCs. Applicants seeded human GSCs on DRG axons grown on coverslips with pre-made parallel scratches as shown in FIG. 2. DRG axon GSC co-cultures were treated with Lck-I or DMSO, which showed that inhibition of Lck results in complete ablation of the finger-like projections consisting of migrating glioma cells that is prominent in control cultures (FIG. 5A). To assess the effect of Lck signaling on the migration and interaction of hGSCs with DRG axons, Applicants seeded hGSCs on DRG axons and treated the cultures with Lck-I or PBS/DMSO for 72 hours. Applicants demonstrated that inhibition of Lck with Lck-I results in significant decrease of GFAP+/Ki67+ migrating tumor cells (FIG. 5B, insets). In all experiments (n=6) treatment with Lck-I resulted in generation of spherical tumor-like structures with limited integration and expansion within the axonal network (FIG. 5B). To quantify the effect of Lck inhibition on GSC migration, Applicants added GSCs on DRG axons with and without Lck-I and quantified the surface area covered by the migrating glioma cells using Image-J.

Applicants demonstrated that inhibition of Lck induces a significant reduction in the total surface area covered by the migrating glioma cells (FIG. 5C, p<0.005). To further analyze the effect of Lck on cell migration, Applicants used the xCELLigence System (Roche) that measures total surface area covered by cell membrane by detecting the electrical impedance, using a microelectronic biosensor technology (Diemert et al., 2012). Inhibition of Lck activity induces significant reduction of human GSC migration (FIG. 5D, p<0.005) indicating an essential role of Lck signaling for human glioma cell migration. To assess the role of Lck signaling on human GSC invasion, Applicants used an Alveltex Scaffold (ReproCELL, Inc), which allows for quantification of cellular invasion in three dimensions (D). Culturing of human GSCs on the D scaffolds with or without the Lck-I shows that inhibition of Lck activity results in significant reduction of GSC invasion as measured by the total area of invasion within the D scaffold using z-stack confocal images (FIG. 6A, p<0.05).

### Example 7—In vivo Administration of Lck-I in an Orthotopic Xenograft Mouse Model of Human Glioblastoma Results in Significant Inhibition of Tumor Formation

To determine the effect of inhibition of Lck signaling in human glioma formation in vivo, Applicants used an orthotopic xenograft mouse model of human glioblastoma (Singh et al., 2004; Valadez et al., 2014). Applicants injected under stereotactic guidance 200,000 hGSCs at coordinates −2.0 mm AP and +1.5 mm ML relative to Bregma. The Lck-I or the PBS/DMSO were administered through an Alzet pump in the lateral ventricle continuously for 4 weeks. Brains were formalin fixed and stained for human Ki67 4 weeks after the implantation. Applicants demonstrated that inhibition of Lck with the highly specific Lck-I induces significant inhibition of tumor formation as compared to animals treated with PBS/DMSO (FIG. 6B). All animals treated with the Lck-I (n=5) showed lack of solid tumor formation, lack of focal necrosis, and minimal invasion and migration of cancer cells within the brain parenchyma. In addition, the local administration of Lck-I had no apparent side effects and the body condition score of all treated animals was excellent. These unique attributes suggest that specific inhibition of Lck signaling is a novel treatment for human glioblastomas.

### Experimental Procedures

### Procedure 1—Isolation and Culture of GBM Stem Cells

The institutional review board at Geisinger Clinic approved the collection and propagation of patient-derived Glioblastoma Multiforme (GBM) tissue through cell culture. Primary GBM neurospheres were cultured from fresh human GBM tumor samples as previously described (1, 2). Contaminating red blood cells were removed from cultures with ACK Lysing Buffer following the manufacturer protocol (Life Technologies). Neurospheres were maintained in culture with Neurobasal-A (1X, Life Technologies), B27-A (1X, Life Technologies), Glutamax (1X, Life Technologies), human EGF and basic FGF (20 ng/ml each, Peprotech) and heparin (2 ug/ml, StemCell Technologies). Media was refreshed to the cultures twice a week. Neurospheres were passaged by mechanical dissociation, aided by TrypLE or StemPro Accutase (Life Technologies) as needed, when they reached a size between 200-500 μM.

### Procedure 2—Self-Renewal Assay

Neurospheres were mechanically dissociated into a single cell suspension and counted. Single cells were seeded in 96-well plates at a density of 1 cell per 100 μl of media. All wells were refreshed with 100 μl of media weekly and were monitored for up to 4 weeks for formation of neurospheres.

### Procedure —Subtype Determination

The subtype of each human primary GBM stem cell line was determined after confirming the presence of subtype-specific gene mutations with qPCR. Genomic DNA from various GBM stem cell lines and H9 human neural stem cells was extracted using the QlAamp DNA Mini Kit (Qiagen) and amplified with the REPLI-G Mini Kit (Qiagen). Quantitative PCR was performed using the Human Brain Cancer qBiomarker Somatic Mutation PCR Array (Qiagen). Relative DNA levels were normalized to GAPDH. Mutations were quantified using the comparative Cₜ method and fold change was calculated versus H9 human neural stem cell control.

### Procedure 4—Dorsal Root Ganglia Cultures

Dorsal root ganglia (DRG) were isolated from E16 rat embryos and cultured in compartmented Campenot chambers as previously described (Campenot, 1982; Chan et al., 2004). Cultures were maintained in media supplemented with NGF and alternating antimitotic agents AraC (2 μM/ml, Sigma) and FUDR (10 μM/ml, Sigma). Axons reached the end of each chamber after two weeks, at which time axons were seeded with oligodendrocyte progenitor cells (OPCs).

Procedure 5—Oligodendrocyte—DRG Neuron Co-Cultures

The cortices from post-natal day P2 rat pups were dissected and diced with a scalpel followed by dissociation by papain (Worthington) and DNase I (Sigma) at 37° C. for 80 minutes. Papain buffer was removed and tissue was triturated in media containing 10% FBS three times, until completely dissociated. Cells were pelleted and resuspended in DMEM including 0.5% BSA and ITS (Life Technologies), filtered through a 30 μm mesh filter, then incubated at 37° C. for 15 min on a non-cell-culture treated 100 mm dish to allow microglia to attach. Floating cells were collected, centrifuged, and anti-A2B5-magnetic bead labeling was conducted per manufacturer's protocol (Miltenyi Biotec). Briefly, cells were incubated with anti-A2B5 microbeads, and the A2B5+ cells were separated and washed with a magnetic column. The isolated cells were resuspended in N2B2 media and seeded on rat DRG neurons and maintained in N2B2+T3 media for 10-14 days to allow for myelination.

### Procedure 6—Lentivirus production

The pLVX-AcGFP1-N1 lentiviral expression vector was purchased from Clontech Laboratories, Inc. and transformed into Top10 chemically competent cells (Life Technologies). Lentivirus was packaged using the Lenti-X 293T cell line (Clontech) and harvested when a high titer was indicated using Lenti-X Go Stix (Clontech). Virus titer was determined using Clontech's Lenti-X qRT-PCR Titration kit. Primary glioblastoma neurospheres from Donor 1 were dissociated, and 300,000 cells were transduced with LentiGFP virus following Clontech's recommendations.

### Procedure 7—Immunocytochemistry

GBM stem cells were cultured on coverslips coated with fibronectin (10 ug/ml, Sigma) and grown in Neurosphere media or Neurosphere media with growth factors removed and supplemented with 10% FBS. Cultures were fixed in 4% formaldehyde after 4 days. The samples were permeabilized and blocked with 5% normal goat serum containing 0.1% Triton X-100 in 1×PBS (Sigma) at room temperature for 1 hour. The samples were stained with the following primary antibodies according to manufacturer specifications overnight at 4° C.: Nestin (Millipore), GFAP (DAKO), MAP2 (Covance) and A2B5 (R&D Systems). The following day, secondary antibodies (Jackson ImmunoResearch) were applied at the recommended dilution for 1 hour. Coverslips were mounted with VectaShield Mounting Medium containing DAPI (Vector Labs) and examined using a Zeiss Axiovert inverted fluorescent microscope.

To assess pseudopodia formation, GBM stem cells were seeded on fibronectin coated (10 ug/ml, Sigma) glass chamber slides (NUNC) in the presence of serum for 2.5 hours. Cultures were treated with 500 nM of Lck inhibitor or DMSO vehicle for 2 hours and then fixed in 4% formaldehyde. Samples were permeabilized and blocked with 5% normal goat serum containing 0.3% Triton X-100 in 1×PBS (Sigma) at RT for 1 hour. The samples were stained overnight at 4° C. with Phospho-Paxillin (CellSignaling). The following day, secondary antibodies were applied at manufacturer recommended dilutions for 1 hour at RT (Jackson ImmunoResearch). Samples were subsequently stained with Rhodamine RedX Phalloidin (Life Technologies) and Hoescht 33342 (Life Technologies) following manufacturer's protocols. Slides were mounted with Aqua Poly/Mount (Polysciences) and imaged using a Zeiss confocal microscope. Adobe Photoshop was used to count the total number of pseudopodia, which included both axial and radial projections, per cell for each group. Differences between groups were confirmed using Student's t-test.

To assess the effect of the Lck inhibitor on migrating glioma cells, GBM spheres were seeded on rat DRG cultures in Supplemented NB-A Medium containing 10% FBS. Spheres were allowed to attach for one hour. Cultures were treated with 500 nM of Lck inhibitor or DMSO vehicle for 72 hours. Samples were fixed in 4% formaldehyde for 10 minutes, permeabilized and blocked with 5% normal goat serum containing 0.3% Triton X-100 in 1×PBS (Sigma) at RT for 1 hour. The samples were stained overnight at 4° C. with Ki67 (Cell Signaling), GFAP (DAKO) and NF (EnCor Biotechnology). Area of glioma stem cell migration for each sample was calculated by normalizing the total area of migration to the interior area of the non-migrating sphere mass. Differences between groups were then confirmed using Student's test.

### Procedure 8—Immunohistochemistry

Tissue microarrays containing paraffin embedded samples of normal brain, glioblastoma multiforme, astrocytoma, oligodendroglioma and meningioma tissues were obtained from US Biomax, Inc. The tissue microarrays were deparaffinized and antigen retrieval was accomplished with sodium citrate buffer. Tissue sections were incubated overnight with Phospho-Lck (Y394) at a concentration of 10 μg/ml (R&D Systems). The following day, tissue microarrays were washed, incubated with ImmPRESS Anti-Mouse/Rabbit IgG Peroxidase (Vector Labs), stained with ImmPACT DAB (Vector Labs) and counterstained with hemotoxylin. Images were captured with a Zeiss Axiovert inverted microscope equipped with a color camera. Photomicrographs were taken at 20× magnification and analyzed for positive staining.

### Procedure 9—Immunoprecipitation and Western Blotting

For immunoprecipitation studies, U87 MG cells treated with A77 or DMSO vehicle were lysed in 1×RIPA buffer containing protease and phosphatase inhibitors (Sigma). Lysates were cleared via centrifugation and were pre-cleared with TrueBlot anti-rabbit IgG beads (eBiosience) to remove non-specific binding. Primary antibodies against Lck, Src, Fyn, Lyn or Yes (Cell Signaling) were added and the lysates were incubated overnight at 4° C. with gentle rotation. The following day, TrueBlot anti-rabbit IgG (eBioscience) beads were added to the lysate and incubated at 4° C. for 2 hours. Immunoprecipitates were washed, solubilized in 2×Laemmli buffer and boiled for 5 minutes. The supernatants were separated on an 8% Bis-Tris SDS-PAGE gel, transferred to nitrocellulose and immunoblotted.

For total cell lysates, cultures were lysed in 1×SDS lysis buffer supplemented with protease and phosphatase inhibitors (Sigma). Lysates were sonicated and cleared by centrifugation. Samples were separated on 4-12% Bis-Tris SDS-PAGE gels (NuPAGE), transferred on to nitrocellulose and immunoblotted using the following primary antibodies: p(Y418)-Src family, Lck (D88), p(Y188)-paxillin, pCrkII (Cell Signaling), mortalin (a kind gift of Dr. Alice Cavanaugh), and β-actin (Sigma). A custom phospho-specific polyclonal antibody was generated against p(Y394)-Lck and affinity-purified against total Lck, Fyn and Lyn (Thermo Fisher). Secondary antibodies used were HRP-conjugate goat anti-mouse and goat anti-rabbit (Cell Signaling). Semi-quantitative analysis was conducted with FluorChem SP analytical software. Statistical significance was determined by the two-tailed Student's t-test.

### Procedure 10—xCELLigence

The xCELLigence system was used to perform cell migration assays on the RTCA DP instrument. Electrical impedance due to cell migration is represented as cell index. GBM cells were seeded on fibronectin coated wells of the RTCA CIM-plate 16 (ACEA Biosciences) and allowed to attach and spread for 1 hour at room temperature. After 1 hour, wells were treated with DMSO vehicle or 500 nM Lck inhibitor. As cells migrated towards the lower chamber containing 10% FBS with DMSO vehicle or 500 nM Lck inhbitor, measurements were captured every 5 minutes. Cell index data was plotted overtime and the slope between 2 and 6 hours was calculated.

### Procedure 11—D-Alvetex Scaffold

Alvetex scaffolds were coated with fibronectin (10 μg/ml) for 2 hours at 37° C. 750,000 GBM cells were plated in the center of each scaffold and allowed to attach for one hour. Cultures were grown in Supplemented NB-A Medium containing 10% FBS. Cultures were treated with 500 nM of Lck inhibitor or DMSO vehicle for 72 hours. Samples were fixed in 4% formaldehyde for 10 minutes, permeabilized with 0.1% Triton X-100 and blocked with 1% BSA-PBS for 30 minutes at room temperature. Samples were subsequently stained with Rhodamine RedX Phalloidin (Life Technologies) and Hoescht 33342 (Life Technologies) following manufacturer's protocols. Slides were mounted with Aqua Poly/Mount (Polysciences) and imaged using a Zeiss confocal microscope.

### Procedure 12—Cell Motility pEIF4E RNA/Local Translation

Local translation of human GBM stem cell pseudopodia cell motility-associated RNA transcripts was confirmed by eIF4E immunoprecipitation as described by Peritz et al. (2006). GBM stem cells (7.5×10⁵) were seeded on 6-well inserts containing 1 μm pores (BD Falcon) coated with 10 ug/nnL human fibronectin (Sigma). After 24 hrs, the sides of the inserts containing only GBM stem cell pseudopodia were washed with ice cold PBS containing 100 ug/mL cycloheximide (Sigma) and scraped directly into polysome lysis buffer (100 mM KCl, 5 mM MgCl₂, 10 mM HEPES, pH 7.0, 0.5% NP-40, 1 mM DTT, 100 U mL⁻¹ RNaseOUT (Life Technologies)). Lysates were centrifuged at 16000 g for 15 min at 4° C., and the antigen-containing supernatants were pre-cleared twice using end-over-end rotation for 1 hour at 4° C. with 50 μl of protein A/G-agarose beads (Santa Cruz) followed by centrifugation at 1000 g for 5 minutes at 4° C. Pre-cleared supernatant was then divided into two equal 1 mL aliquots and incubated with either 10 ug eIF4E antiserum (Santa Cruz) or no antiserum overnight at 4° C. The following day each aliquot was rotated in the presence of 50 μl protein A/G-agarose beads for 4 hours at 4° C. After briefly centrifuging to collect the beads from each aliquot, beads were washed 4 times by adding 0.5 mL polysome lysis buffer and rotating for 5 min at 4° C. followed by centrifugation at 1000 g for 5 minutes at 4° C. Beads were washed 4 more times as described previously but with polysome lysis buffer containing 1M urea. They were then separated from the RNA by resuspending in 100 μl of polysome lysis buffer with 0.1% SDS and 30 ug proteinase K (Ambion) and incubating at 50° C. for 30 minutes. RNA was extracted with phenol-chloroform-isoamyl alcohol mixture (Ambion), vortexed, and centrifuged at maximum speed at RT for 1 min, and the entire separation/extraction procedure was repeated once before overnight precipitation in ethanol. Precipitated RNA was treated with 10 U of DNase I (NEB) for 15 min at 37° C., extracted, precipitated, and dissolved in 10 μL of nuclease-free water. Immunoprecipitated RNA samples were reverse-transcribed using the RTZ First Strand Kit (Qiagen) and 100 ng of each cDNA aliquot was pre-amplified using RTZ PreAMP cDNA Sythenthesis Primer Mix for Human Cell Motility PCR Array as well as Lck and Crk-specific primers. Quantitative PCR was performed using the Human Cell Motility RT² PCR Array (Qiagen) and separately with Lck and Crk-specific primers (Qiagen). Relative DNA levels were normalized to pre-amplified GAPDH levels in whole neurosphere controls. Amplifications of p-eIF4E bound and nonspecific immunoprecipitated cell motility-associated transcripts were quantified using the comparative Cₜ method and fold change was calculated versus nonspecific immunoprecipitated controls.

The presence of human cell motility-associated RNA transcripts within the GBM stem cell pseudopodia was determined via qRT-PCR. Total RNA was isolated from pseudopodia that had migrated through 1 μM pores of 10 ug/mL human fibronectin-coated 6-well inserts (BD Falcon) for 24 hours. Total RNA from each GBM stem cell pseudopodia sample was reverse-transcribed using the RT² First Strand Kit (Qiagen). Quantitative PCR was performed using the Human Cell Motility RT² PCR Array (Qiagen). The relative abundance of each cell motility-associated pseudopodia RNA transcript (Cₜ<35) was collectively represented by generating a gene heat map from GAPDH normalized Ct values with GENE-E software (Broad Institute).

### Procedure 13—Stereotactic Injections

Intracranial injections were performed using a stereotaxic apparatus (Kopf) on 8-week-old Nu/J mice initially sedated with 4% isoflurane and maintained with 2% isoflurane. After leveling the skull, a hole was drilled with a #72 micro drill bit (Kyocera) at coordinates −2.0 mm AP and +1.5 mm ML relative to Bregma. A 75 RN Hamilton syringe was then lowered to a depth of −2.5 mm DV at a rate of 0.5 mm per minute, and 200,000 primary GSCs from donor 2 resuspended in a total volume of 4 μl were injected at a rate of 0.5 μl per minute using a Stoelting Quintessiential Stereotaxic Injector. In order to reduce backflow, the syringe was allowed to rest for 2 minutes post-injection before it was withdrawn at a rate of 0.5 mm per minute, and the cavity was immediately sealed with bone wax (Ethicon). A subcutaneous pocket was formed, and an Alzet osmotic pump 1004 fitted with brain infusion kit 3 and containing either 1.175 mg of A77 (Axon Medchem) or DMSO/Kolliphor ELP vehicle was inserted near the left hind limb of the animal. A second hole was drilled at coordinates of +0.5 mm and +1.1 mm ML relative to Bregma with the same micro drill bit to allow placement of the brain infusion catheter in the right lateral ventricle. The brain infusion catheter was lowered into the brain and glued to the skull using Loctite 454 cyanoacrylate adhesive. The incision was sealed using veterinary tissue glue and a dab of antibiotic ointment was placed over the head and neck. Animals were monitored daily for weight loss, pain and distress. After four weeks, animals were euthanized with pentobarbital and perfused with 4% formaldehyde. Brains were harvested, post-fixed in 4% formaldehyde, and processed for paraffin embedding. Paraffin embedded tissue was sliced on a microtome to 5 μM sections and placed on microscope slides. Tissues were stained for H&E and ki67 (Roche) using a Ventana Autostainer. Images were captured using a Zeiss Axiovert inverted microscope.

### Procedure 14—Statistical analysis

Quantification of cellular migration, invasion and pseudopodia formation was performed using unpaired student's t-test. Densitometric quantification of Western blots was performed with paired student's t-test. In all cases significance was determined when p<0.05.

### REFERENCES

Each of the following references is incorporated by reference herein in its entirety for all purposes:
Adilakshmi, T., Ness-Myers, J., Madrid-Aliste, C., Fiser, A., and Tapinos, N. (2011). A nuclear variant of ErbB3 receptor tyrosine kinase regulates ezrin distribution and Schwann cell myelination. J Neurosci 31, 5106-5119.
Aubert, M., Badoual, M., Christov, C., and Grammaticos, B. (2008). A model for glioma cell migration on collagen and astrocytes. Journal of the Royal Society, Interface/the Royal Society 5, 75-83.
Bao, S., Wu, Q., McLendon, R. E., Hao, Y., Shi, Q., Hjelmeland, A. B., Dewhirst, M. W., Bigner, D. D., and Rich, J. N. (2006). Glioma stem cells promote radioresistance by preferential activation of the DNA damage response. Nature 444, 756-760.
Bedard, P. L., Hansen, A. R., Ratain, M. J., and Siu, L. L. (2013). Tumour heterogeneity in the clinic. Nature 501, 355-364.
Beier, D., Hau, P., Proescholdt, M., Lohmeier, A., Wischhusen, J., Oefner, P. J., Aigner, L., Brawanski, A., Bogdahn, U., and Beier, C. P. (2007). CD133(+) and CD133(−) glioblastoma-derived cancer stem cells show differential growth characteristics and molecular profiles. Cancer Res 67, 4010-4015.
Brennan, C. W., Verhaak, R. G., McKenna, A., Campos, B., Noushmehr, H., Salama, S. R., Zheng, S., Chakravarty, D., Sanborn, J. Z., Berman, S. H., et al. (2013). The somatic genomic landscape of glioblastoma. Cell 155, 462-477.
Burchat, A., Borhani, D. W., Calderwood, D. J., Hirst, G. C., Li, B., and Stachlewitz, R. F. (2006). Discovery of A-770041, a src-family selective orally active Ick inhibitor that prevents organ allograft rejection. Bioorg Med Chem Lett 16, 118-122.
Campenot, R. B. (1982). Development of sympathetic neurons in compartmentalized cultures. II Local control of neurite growth by nerve growth factor. Dev Biol 93, 1-12. Cancer Genome Atlas Research, N. (2008). Comprehensive genomic characterization defines human glioblastoma genes and core pathways. Nature 455, 1061-1068.
Chan, J. R., Watkins, T. A., Cosgaya, J. M., Zhang, C., Chen, L., Reichardt, L. F., Shooter, E. M., and Barres, B. A. (2004). NGF controls axonal receptivity to myelination by Schwann cells or oligodendrocytes. Neuron 43, 183-191.
Cho, S. Y., and Klemke, R. L. (2002). Purification of pseudopodia from polarized cells reveals redistribution and activation of Rac through assembly of a CAS/Crk scaffold. J Cell Biol 156, 725-736.
de la Iglesia, N., Puram, S. V., and Bonni, A. (2009). STAT3 regulation of glioblastoma pathogenesis. Curr Mol Med 9, 580-590.
Deakin, N. O., and Turner, C. E. (2008). Paxillin comes of age. J Cell Sci 121, 2435-2444.
Demuth, T., and Berens, M. E. (2004). Molecular mechanisms of glioma cell migration and invasion. Journal of neuro-oncology 70, 217-228.
Diemert, S., Dolga, A. M., Tobaben, S., Grohm, J., Pfeifer, S., Oexler, E., and Culmsee, C. (2012). Impedance measurement for real time detection of neuronal cell death. Journal of neuroscience methods 203, 69-77.
Dugas, J. C., Tai, Y. C., Speed, T. P., Ngai, J., and Barres, B. A. (2006). Functional genomic analysis of oligodendrocyte differentiation. J Neurosci 26, 10967-10983.
Friedl, P., and Wolf, K. (2003). Tumour-cell invasion and migration: diversity and escape mechanisms. Nat Rev Cancer , 362-374.
Galli, R., Binda, E., Orfanelli, U., Cipelletti, B., Gritti, A., De Vitis, S., Fiocco, R., Foroni, C., Dimeco, F., and Vescovi, A. (2004). Isolation and characterization of tumorigenic, stem-like neural precursors from human glioblastoma. Cancer Res 64, 7011-7021.
Giese, A., Kluwe, L., Laube, B., Meissner, H., Berens, M. E., and Westphal, M. (1996). Migration of human glioma cells on myelin. Neurosurgery 38, 755-764.
Han, X., Zhang, W., Yang, X., Wheeler, C. G., Langford, C. P., Wu, L., Filippova, N., Friedman, G. K., Ding, Q., Fathallah-Shaykh, H. M., et al. (2014). The role of Src family kinases in growth and migration of glioma stem cells. International journal of oncology 45, 302-310.
Hynes, R. O. (2007). Cell-matrix adhesion in vascular development. J Thromb Haemost 5 Suppl 1, 32-40.
Ishigaki, Y., Li, X., Serin, G., and Maquat, L. E. (2001). Evidence for a pioneer round of mRNA translation: mRNAs subject to nonsense-mediated decay in mammalian cells are bound by CBP80 and CBP20. Cell 106, 607-617.
Joy, A. M., Beaudry, C. E., Tran, N. L., Ponce, F. A., Holz, D. R., Demuth, T., and Berens, M. E. (2003). Migrating glioma cells activate the P13-K pathway and display decreased susceptibility to apoptosis. J Cell Sci 116, 4409-4417.
Jung, H., Gkogkas, C. G., Sonenberg, N., and Holt, C. E. (2014). Remote control of gene function by local translation. Cell 157, 26-40.
Kaur, H., Phillips-Mason, P. J., Burden-Gulley, S. M., Kerstetter-Fogle, A. E., Basilion, J. P., Sloan, A. E., and Brady-Kalnay, S. M. (2012). Cadherin-11, a marker of the mesenchymal phenotype, regulates glioblastoma cell migration and survival in vivo. Mol Cancer Res 10, 293-304.
Kim, R. K., Yoon, C. H., Hyun, K. H., Lee, H., An, S., Park, M. J., Kim, M. J., and Lee, S. J. (2010). Role of lymphocyte-specific protein tyrosine kinase (LCK) in the expansion of glioma-initiating cells by fractionated radiation. Biochem Biophys Res Commun 402, 631-636.
Kislin, K. L., McDonough, W. S., Eschbacher, J. M., Armstrong, B. A., and Berens, M. E. (2009). NHERF-1: modulator of glioblastoma cell migration and invasion. Neoplasia 11, 377-387.
Koul, D., Shen, R., Bergh, S., Lu, Y., de Groot, J. F., Liu, T. J., Mills, G. B., and Yung, W. K. (2005). Targeting integrin-linked kinase inhibits Akt signaling pathways and decreases tumor progression of human glioblastoma. Mol Cancer Ther 4, 1681-1688.
Lassman, A. B., Pugh, S. L., Gilbert, M. R., Aldape, K. D., Geinoz, S., Beumer, J. H., Christner, S. M., Komaki, R., DeAngelis, L. M., Gaur, R., et al. (2015). Phase 2 trial of dasatinib in target-selected patients with recurrent glioblastoma (RTOG 0627). Neuro-oncology.
Lee, J., Kotliarova, S., Kotliarov, Y., Li, A., Su, Q., Donin, N. M., Pastorino, S., Purow, B. W., Christopher, N., Zhang, W., et al. (2006). Tumor stem cells derived from glioblastomas cultured in bFGF and EGF more closely mirror the phenotype and genotype of primary tumors than do serum-cultured cell lines. Cancer Cell 9, 391-403.
Leung, K. M., van Horck, F. P., Lin, A. C., Allison, R., Standart, N., and Holt, C. E. (2006). Asymmetrical beta-actin mRNA translation in growth cones mediates attractive turning to netrin-1. Nat Neurosci 9, 1247-1256.
MacDonald, T. J., Taga, T., Shimada, H., Tabrizi, P., Zlokovic, B. V., Cheresh, D. A., and Laug, W. E. (2001). Preferential susceptibility of brain tumors to the antiangiogenic effects of an alpha(v) integrin antagonist. Neurosurgery 48, 151-157.
Merzak, A., and Pilkington, G. J. (1997). Molecular and cellular pathology of intrinsic brain tumours. Cancer Metastasis Rev 16, 155-177.
Mili, S., Moissoglu, K., and Macara, I. G. (2008). Genome-wide screen reveals APC-associated RNAs enriched in cell protrusions. Nature 453, 115-119.
Milinkovic, V., Bankovic, J., Rakic, M., Stankovic, T., Skender-Gazibara, M., Ruzdijic, S., and Tanic, N. (2013). Identification of novel genetic alterations in samples of malignant glioma patients. PLoS One 8, e82108.
Morales, F. C., Molina, J. R., Hayashi, Y., and Georgescu, M. M. (2010). Overexpression of ezrin inactivates NF2 tumor suppressor in glioblastoma. Neuro-oncology 12, 528-539.
Ness, J. K., Snyder, K. M., and Tapinos, N. (2013). Lck tyrosine kinase mediates betal-integrin signalling to regulate Schwann cell migration and myelination. Nature communications 4, 1912.
Park, J. B., Agnihotri, S., Golbourn, B., Bertrand, K. C., Luck, A., Sabha, N., Smith, C. A., Byron, S., Zadeh, G., Croul, S., et al. (2014). Transcriptional profiling of GBM invasion genes identifies effective inhibitors of the LIM kinase-Cofilin pathway. Oncotarget 5, 9382-9395.
Patel, A. P., Tirosh, I., Trombetta, J. J., Shalek, A. K., Gillespie, S. M., Wakimoto, H., Cahill, D. P., Nahed, B. V., Curry, W. T., Martuza, R. L., et al. (2014). Single-cell RNA-seq highlights intratumoral heterogeneity in primary glioblastoma. Science 344,1396-1401.
Piper, M., Anderson, R., Dwivedy, A., Weinl, C., van Horck, F., Leung, K. M., Cogill, E., and Holt, C. (2006). Signaling mechanisms underlying Slit2-induced collapse of Xenopus retinal growth cones. Neuron 49, 215-228.
Rambukkana, A., Zanazzi, G., Tapinos, N., and Salzer, J. L. (2002). Contact-dependent demyelination by Mycobacterium leprae in the absence of immune cells. Science 296, 927-931.
Rao, S. S., Nelson, M. T., Xue, R., DeJesus, J. K., Viapiano, M. S., Lannutti, J. J., Sarkar, A., and Winter, J. O. (2013). Mimicking white matter tract topography using core-shell electrospun nanofibers to examine migration of malignant brain tumors. Biomaterials 34, 5181-5190.
Reyes, S. B., Narayanan, A. S., Lee, H. S., Tchaicha, J. H., Aldape, K. D., Lang, F. F., Tolias, K. F., and McCarty, J. H. (2013). alphavbeta8 integrin interacts with RhoGDI1 to regulate Rac1 and Cdc42 activation and drive glioblastoma cell invasion. Mol Biol Cell 24, 474-482.
Reynolds, P. J., Hurley, T. R., and Sefton, B. M. (1992). Functional analysis of the SH2 and SH3 domains of the Ick tyrosine protein kinase. Oncogene 7, 1949-1955.
Ruffini, F., Arbour, N., Blain, M., Olivier, A., and Antel, J. P. (2004). Distinctive properties of human adult brain-derived myelin progenitor cells. The American journal of pathology 165, 2167-2175.
Singh, S. K., Clarke, I. D., Terasaki, M., Bonn, V. E., Hawkins, C., Squire, J., and Dirks, P. B. (2003). Identification of a cancer stem cell in human brain tumors. Cancer Res 63, 5821-5828.
Singh, S. K., Hawkins, C., Clarke, I. D., Squire, J. A., Bayani, J., Hide, T., Henkelman, R. M., Cusimano, M. D., and Dirks, P. B. (2004). Identification of human brain tumour initiating cells. Nature 432,396-401.
Stachlewitz, R. F., Hart, M. A., Bettencourt, B., Kebede, T., Schwartz, A., Ratnofsky, S. E., Calderwood, D. J., Waegell, W. O., and Hirst, G. C. (2005). A-770041, a novel and selective small-molecule inhibitor of Lck, prevents heart allograft rejection. J Pharmacol Exp Ther 315, 36-41.
Stupp, R., Mason, W. P., van den Bent, M. J., Weller, M., Fisher, B., Taphoorn, M. J., Belanger, K., Brandes, A. A., Marosi, C., Bogdahn, U., et al. (2005). Radiotherapy plus concomitant and adjuvant temozolomide for glioblastoma. N Engl J Med 352, 987-996.
Stupp, R., and Weber, D. C. (2005). The role of radio- and chemotherapy in glioblastoma. Onkologie 28, 315-317.
Taga, T., Suzuki, A., Gonzalez-Gomez, I., Gilles, F. H., Stins, M., Shimada, H., Barsky, L., Weinberg, K. I., and Laug, W. E. (2002). alpha v-Integrin antagonist EMD 121974 induces apoptosis in brain tumor cells growing on vitronectin and tenascin. Int J Cancer 98, 690-697.
Tapinos, N., Ohnishi, M., and Rambukkana, A. (2006). ErbB2 receptor tyrosine kinase signaling mediates early demyelination induced by leprosy bacilli. Nat Med 12, 961-966.
Tonn, J. C., Wunderlich, S., Kerkau, S., Klein, C. E., and Roosen, K. (1998). Invasive behaviour of human gliomas is mediated by interindividually different integrin patterns. Anticancer Res 18, 2599-2605.
Valadez, J. G., Sarangi, A., Lundberg, C. J., and Cooper, M. K. (2014). Primary orthotopic glioma xenografts recapitulate infiltrative growth and isocitrate dehydrogenase I mutation. Journal of visualized experiments: JoVE, e50865.
Verhaak, R. G., Hoadley, K. A., Purdom, E., Wang, V., Qi, Y., Wilkerson, M. D., Miller, C. R., Ding, L., Golub, T., Mesirov, J. P., et al. (2010). Integrated genomic analysis identifies clinically relevant subtypes of glioblastoma characterized by abnormalities in PDGFRA, IDH1, EGFR, and NF1. Cancer Cell 17, 98-110.
Vescovi, A. L., Galli, R., and Reynolds, B. A. (2006). Brain tumour stem cells. Nat Rev Cancer 6, 425-436.
Wang, Y., Ding, S. J., Wang, W., Yang, F., Jacobs, J. M., Camp, D., 2nd, Smith, R. D., and Klemke, R. L. (2007). Methods for pseudopodia purification and proteomic analysis. Science's STKE: signal transduction knowledge environment 2007, p 14.
Wang, Y., and Klemke, R. L. (2007). Biochemical purification of pseudopodia from migratory cells. Methods in molecular biology (Clifton, N.J. 370, 55-66.
Wick, W., Grimmel, C., Wild-Bode, C., Platten, M., Arpin, M., and Weller, M. (2001). Ezrin-dependent promotion of glioma cell clonogenicity, motility, and invasion mediated by BCL-2 and transforming growth factor-beta2. J Neurosci 21, 3360-3368.
Windebank, A. J., Wood, P., Bunge, R. P., and Dyck, P. J. (1985). Myelination determines the caliber of dorsal root ganglion neurons in culture. J Neurosci 5, 1563-1569.
Wood, P. M. (1976). Separation of functional Schwann cells and neurons from normal peripheral nerve tissue. Brain research 115, 361-375.
Wu, K. Y., Hengst, U., Cox, L. J., Macosko, E. Z., Jeromin, A., Urquhart, E. R., and Jaffrey, S. R. (2005). Local translation of RhoA regulates growth cone collapse. Nature 436, 1020-1024.
Yamaguchi, H., and Hendrickson, W. A. (1996). Structural basis for activation of human lymphocyte kinase Lck upon tyrosine phosphorylation. Nature 384, 484-489.
Yao, J., Sasaki, Y., Wen, Z., Bassell, G. J., and Zheng, J. Q. (2006). An essential role for beta-actin mRNA localization and translation in Ca2+-dependent growth cone guidance. Nat Neurosci 9, 1265-1273.

### INCORPORATION BY REFERENCE

Various publications are cited herein. The content of each cited publication is incorporated by reference herein in its entirety for all purposes.

1. A method for treating glioblastoma in a subject comprising administering to the subject an inhibitor of a Src family kinase, wherein the inhibitor is not Dasatinib.

2. The method of claim 1, wherein the Src family kinase is lymphocyte-specific protein tyrosine kinase (Lck).

3. The method of claim 1, wherein the inhibitor is a small molecule.

4. The method of claim 1, wherein the inhibitor reduces the level of phosphorylation of the Src family kinase.

5. The method of claim 1, wherein the inhibitor reduces the level of phosphorylation of paxillin.

6. The method of claim 1, wherein the inhibitor is A770041.

7. The method of claim 1, wherein the subject is a human, mouse, rat, or dog.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A method for treating glioblastoma in a subject comprising reducing the expression of a Src family kinase protein in a gliomal cell of the subject.

59. (canceled)

60. (canceled)

61. The method of claim 58, wherein the Src family kinase protein is lymphocyte-specific protein tyrosine kinase (Lck).

62. The method of claim 58, wherein the expression of the Src family kinase protein is reduced by a method comprising administering an antisense oligonucleotide to the gliomal cell of the subject.

63. The method of claim 62, wherein the antisense oligonucleotide targets a transcript encoding the Src family kinase protein.

64. The method of claim 58, wherein the expression of the Src family kinase protein is reduced by a method comprising RNA interference in the gliomal cell of the subject or the glial cell.

65. The method of claim 64, wherein the RNA interference method comprises administering a small interfering RNA (siRNA) to the gliomal cell of the subject or the glial cell.

66. The method of claim 65, wherein the siRNA targets a transcript encoding the Src family kinase protein.

67. The method of claim 64, wherein the RNA interference method comprises administering a small hairpin RNA (shRNA) to the gliomal cell of the subject or the glial cell.

68. The method of claim 67, wherein the shRNA targets a transcript encoding the Src family kinase protein.

69. The method of claim 58, wherein the expression of the Src family kinase protein is reduced by a method comprising gene editing.

70. The method of claim 69, wherein the gene editing method comprises altering the expression of a gene that encodes the Src family kinase protein.

71. The method of claim 70, wherein the gene editing method comprises administering a clustered regularly interspaced short palindromic repeats (CRISPR)/CRISPR-associated (Cas) (CRISPR/Cas) system to the gliomal cell of the subject or the glial cell.

72. The method of claim 71, wherein the CRISPR/Cas system targets a gene that encodes the Src family kinase protein.

73. The method of claim 72, wherein the method comprises a mutation, substitution, and/or deletion in the gene that encodes the Src family kinase protein.
